SEQUENCE LISTING (64 sequences)

SEQ ID NO: 1 - DNA, 19 bp, synthetic construct
  misc_feature 1..19: note = Synthetic primer
  source 1..19: mol_type = other DNA
tgccgtttgtgcttgctag

SEQ ID NO: 2 - DNA, 19 bp, synthetic construct
  misc_feature 1..19: note = Synthetic primer
  source 1..19: mol_type = other DNA
aggtttaccactccctgct

SEQ ID NO: 3 - DNA, 39 bp, synthetic construct
  misc_feature 1..39: note = Synthetic primer
  source 1..39: mol_type = other DNA
atcggatgaaagcctgcggcgccgcaagtacattctggc

SEQ ID NO: 4 - DNA, 42 bp, synthetic construct
  misc_feature 1..42: note = Synthetic primer
  source 1..42: mol_type = other DNA
taaccacgcatttgtcgtccggcaacacgaggcttttcaacc

SEQ ID NO: 5 - DNA, 18 bp, synthetic construct
  misc_feature 1..18: note = Synthetic primer
  source 1..18: mol_type = other DNA
actttcaacgtggtgggc

SEQ ID NO: 6 - DNA, 19 bp, synthetic construct
  misc_feature 1..19: note = Synthetic primer
  source 1..19: mol_type = other DNA
ggctccactacggtcaacg

SEQ ID NO: 7 - DNA, 19 bp, synthetic construct
  misc_feature 1..19: note = Synthetic primer
  source 1..19: mol_type = other DNA
tgccggttgtgcttgctag

SEQ ID NO: 8 - DNA, 19 bp, synthetic construct
  misc_feature 1..19: note = Synthetic primer
  source 1..19: mol_type = other DNA
aggttcatcactccctgct

SEQ ID NO: 9 - DNA, 39 bp, synthetic construct
  misc_feature 1..39: note = Synthetic primer
  source 1..39: mol_type = other DNA
atcggatgaaagcccgcggcgccgcgagtacgttctggc

SEQ ID NO: 10 - DNA, 42 bp, synthetic construct
  misc_feature 1..42: note = Synthetic primer
  source 1..42: mol_type = other DNA
taaccacgcatttgtcgtccggcaacacgaggctttttaacc

SEQ ID NO: 11 - DNA, 18 bp, synthetic construct
  misc_feature 1..18: note = Synthetic primer
  source 1..18: mol_type = other DNA
actttcgacgtggtgggc

SEQ ID NO: 12 - DNA, 19 bp, synthetic construct
  misc_feature 1..19: note = Synthetic primer
  source 1..19: mol_type = other DNA
ggctctactacagttaacg

SEQ ID NO: 13 - DNA, 20 bp, synthetic construct
  misc_feature 1..20: note = Synthetic primer
  source 1..20: mol_type = other DNA
gaacgtggcgataatttcac

SEQ ID NO: 14 - DNA, 19 bp, synthetic construct
  misc_feature 1..19: note = Synthetic primer
  source 1..19: mol_type = other DNA
atttcctgatcgcactgaa

SEQ ID NO: 15 - DNA, 41 bp, synthetic construct
  misc_feature 1..41: note = Synthetic primer
  source 1..41: mol_type = other DNA
cacgctctttcgtctggcattagcatcggcttcaatcaaga

SEQ ID NO: 16 - DNA, 40 bp, synthetic construct
  misc_feature 1..40: note = Synthetic primer
  source 1..40: mol_type = other DNA
acagtaccgcaggaaacgttgtactggcgatattggtgtt

SEQ ID NO: 17 - DNA, 21 bp, synthetic construct
  misc_feature 1..21: note = Synthetic primer
  source 1..21: mol_type = other DNA
cgatcagtaccagtcgtctta

SEQ ID NO: 18 - DNA, 22 bp, synthetic construct
  misc_feature 1..22: note = Synthetic primer
  source 1..22: mol_type = other DNA
aggattctgtcaatgtagaacg

SEQ ID NO: 19 - DNA, 19 bp, synthetic construct
  misc_feature 1..19: note = Synthetic primer
  source 1..19: mol_type = other DNA
acacgttctgaacctttgg

SEQ ID NO: 20 - DNA, 18 bp, synthetic construct
  misc_feature 1..18: note = Synthetic primer
  source 1..18: mol_type = other DNA
taccacgctctttcgtct

SEQ ID NO: 21 - DNA, 41 bp, synthetic construct
  misc_feature 1..41: note = Synthetic primer
  source 1..41: mol_type = other DNA
ttcgggcaattcgttattggcccacggtgacaatagagaag

SEQ ID NO: 22 - DNA, 40 bp, synthetic construct
  misc_feature 1..40: note = Synthetic primer
  source 1..40: mol_type = other DNA
gtggcgataatttcaccggcggcattatcgatcagtacca

SEQ ID NO: 23 - DNA, 17 bp, synthetic construct
  misc_feature 1..17: note = Synthetic primer
  source 1..17: mol_type = other DNA
ggcggtgggttttgttg

SEQ ID NO: 24 - DNA, 22 bp, synthetic construct
  misc_feature 1..22: note = Synthetic primer
  source 1..22: mol_type = other DNA
tcggcttcaatcaagataagac

SEQ ID NO: 25 - DNA, 18 bp, synthetic construct
  misc_feature 1..18: note = Synthetic primer
  source 1..18: mol_type = other DNA
cggcaatagcgtcacctt

SEQ ID NO: 26 - DNA, 18 bp, synthetic construct
  misc_feature 1..18: note = Synthetic primer
  source 1..18: mol_type = other DNA
cggcccgattttctctgg

SEQ ID NO: 27 - DNA, 42 bp, synthetic construct
  misc_feature 1..42: note = Synthetic primer
  source 1..42: mol_type = other DNA
gcgaagcgtactggaaagggaataaacttcatcgcaccgtca

SEQ ID NO: 28 - DNA, 40 bp, synthetic construct
  misc_feature 1..40: note = Synthetic primer
  source 1..40: mol_type = other DNA
gcgcggcatccgcatcaatatgcccggtaaacagatgagt

SEQ ID NO: 29 - DNA, 19 bp, synthetic construct
  misc_feature 1..19: note = Synthetic primer
  source 1..19: mol_type = other DNA
gccagctttacggttcctt

SEQ ID NO: 30 - DNA, 18 bp, synthetic construct
  misc_feature 1..18: note = Synthetic primer
  source 1..18: mol_type = other DNA
gccttcaaatcggcatca

SEQ ID NO: 31 - DNA, 19 bp, synthetic construct
  misc_feature 1..19: note = Synthetic primer
  source 1..19: mol_type = other DNA
tcaacaatgcggggatctg

SEQ ID NO: 32 - DNA, 20 bp, synthetic construct
  misc_feature 1..20: note = Synthetic primer
  source 1..20: mol_type = other DNA
gaagcgtactggaaagggaa

SEQ ID NO: 33 - DNA, 42 bp, synthetic construct
  misc_feature 1..42: note = Synthetic primer
  source 1..42: mol_type = other DNA
tggggatgactcgccatggtatgaccatcaccaatggtcagc

SEQ ID NO: 34 - DNA, 41 bp, synthetic construct
  misc_feature 1..41: note = Synthetic primer
  source 1..41: mol_type = other DNA
aatgatgccggcaatagcgtcagccagctttacggttcctt

SEQ ID NO: 35 - DNA, 20 bp, synthetic construct
  misc_feature 1..20: note = Synthetic primer
  source 1..20: mol_type = other DNA
ccgccctgtctacttatacc

SEQ ID NO: 36 - DNA, 17 bp, synthetic construct
  misc_feature 1..17: note = Synthetic primer
  source 1..17: mol_type = other DNA
acttcatcgcaccgtca

SEQ ID NO: 37 - DNA, 21 bp, synthetic construct
  misc_feature 1..21: note = Synthetic primer
  source 1..21: mol_type = other DNA
cgatcagtaccagccgtctta

SEQ ID NO: 38 - DNA, 19 bp, synthetic construct
  misc_feature 1..19: note = Synthetic primer
  source 1..19: mol_type = other DNA
atcctcccgaagccttttg

SEQ ID NO: 39 - DNA, 41 bp, synthetic construct
  misc_feature 1..41: note = Synthetic primer
  source 1..41: mol_type = other DNA
ttcggtcatttcctggtcggctaatgacgaggatgataaaa

SEQ ID NO: 40 - DNA, 17 bp, synthetic construct
  misc_feature 1..17: note = Synthetic primer
  source 1..17: mol_type = other DNA
cgcggtcgggattgttg

SEQ ID NO: 41 - DNA, 18 bp, synthetic construct
  misc_feature 1..18: note = Synthetic primer
  source 1..18: mol_type = other DNA
cggcaatcgtatcgcctt

SEQ ID NO: 42 - DNA, 18 bp, synthetic construct
  misc_feature 1..18: note = Synthetic primer
  source 1..18: mol_type = other DNA
gcgcccgattcgtgcttg

SEQ ID NO: 43 - DNA, 42 bp, synthetic construct
  misc_feature 1..42: note = Synthetic primer
  source 1..42: mol_type = other DNA
gcgaagcgtccagcaaagggaataaatttcatcgcaccgtcc

SEQ ID NO: 44 - DNA, 40 bp, synthetic construct
  misc_feature 1..40: note = Synthetic primer
  source 1..40: mol_type = other DNA
gggcatcatctgcaccgatctgcccggtaaacagatgagt

SEQ ID NO: 45 - DNA, 19 bp, synthetic construct
  misc_feature 1..19: note = Synthetic primer
  source 1..19: mol_type = other DNA
gccagctttacggtgcgat

SEQ ID NO: 46 - DNA, 19 bp, synthetic construct
  misc_feature 1..19: note = Synthetic primer
  source 1..19: mol_type = other DNA
tcaacaatgccgggatctg

SEQ ID NO: 47 - DNA, 20 bp, synthetic construct
  misc_feature 1..20: note = Synthetic primer
  source 1..20: mol_type = other DNA
gaagcgtccagcaaagggaa

SEQ ID NO: 48 - DNA, 42 bp, synthetic construct
  misc_feature 1..42: note = Synthetic primer
  source 1..42: mol_type = other DNA
tggggatgacccgccatggtatggccgtcgccaatggtcagc

SEQ ID NO: 49 - DNA, 41 bp, synthetic construct
  misc_feature 1..41: note = Synthetic primer
  source 1..41: mol_type = other DNA
aataataccggcaatagcgtcagccagctttacggtgcgat

SEQ ID NO: 50 - DNA, 20 bp, synthetic construct
  misc_feature 1..20: note = Synthetic primer
  source 1..20: mol_type = other DNA
ccgctctgtctacttacacc

SEQ ID NO: 51 - DNA, 17 bp, synthetic construct
  misc_feature 1..17: note = Synthetic primer
  source 1..17: mol_type = other DNA
atttcatcgcaccgtcc

SEQ ID NO: 52 - DNA, 20 bp, synthetic construct
  misc_feature 1..20: note = Synthetic primer
  source 1..20: mol_type = other DNA
caggcgattaaagagctgat

SEQ ID NO: 53 - DNA, 19 bp, synthetic construct
  misc_feature 1..19: note = Synthetic primer
  source 1..19: mol_type = other DNA
tgatggtatcggtgtgagc

SEQ ID NO: 54 - DNA, 39 bp, synthetic construct
  misc_feature 1..39: note = Synthetic primer
  source 1..39: mol_type = other DNA
ggacgggtatccggttcgtttgacaaaaaccacccaagc

SEQ ID NO: 55 - DNA, 40 bp, synthetic construct
  misc_feature 1..40: note = Synthetic primer
  source 1..40: mol_type = other DNA
acgggaatatttcgcgccacttacattgacgcaggtgatc

SEQ ID NO: 56 - DNA, 19 bp, synthetic construct
  misc_feature 1..19: note = Synthetic primer
  source 1..19: mol_type = other DNA
ggcggaagcaacgcgtaaa

SEQ ID NO: 57 - DNA, 20 bp, synthetic construct
  misc_feature 1..20: note = Synthetic primer
  source 1..20: mol_type = other DNA
caggcgattaacgagttgat

SEQ ID NO: 58 - DNA, 19 bp, synthetic construct
  misc_feature 1..19: note = Synthetic primer
  source 1..19: mol_type = other DNA
tgatggtatcggcgtgagc

SEQ ID NO: 59 - DNA, 19 bp, synthetic construct
  misc_feature 1..19: note = Synthetic primer
  source 1..19: mol_type = other DNA
ttagcgggcaattcatctc

SEQ ID NO: 60 - DNA, 19 bp, synthetic construct
  misc_feature 1..19: note = Synthetic primer
  source 1..19: mol_type = other DNA
gtgagggcttctgtctttg

SEQ ID NO: 61 - DNA, 39 bp, synthetic construct
  misc_feature 1..39: note = Synthetic primer
  source 1..39: mol_type = other DNA
acaaacacatcccccctggaatttgccccattagcggat

SEQ ID NO: 62 - DNA, 41 bp, synthetic construct
  misc_feature 1..41: note = Synthetic primer
  source 1..41: mol_type = other DNA
ctcatgctcccacttggaatgcccattggagtgcttgtcat

SEQ ID NO: 63 - DNA, 24 bp, synthetic construct
  misc_feature 1..24: note = Synthetic primer
  source 1..24: mol_type = other DNA
ccgatccttatactgttgtcctta

SEQ ID NO: 64 - DNA, 19 bp, synthetic construct
  misc_feature 1..19: note = Synthetic primer
  source 1..19: mol_type = other DNA
actcagggagccttgctga

(19) United States
(12) Patent Application Publication
(10) Pub. No.: US 2024/0271228 A1
(43) Pub. Date: Aug. 15, 2024

(54) COMPOSITIONS, DEVICES AND METHODS FOR AN IMPROVED RAPID POINT-OF-USE DIAGNOSTIC ASSAY

(71) Applicant: HYPERCELL TECHNOLOGIES, Peachtree Corners, GA (US)

(72) Inventors: Bruno Georges JACTEL, Peachtree Corners, GA (US); Alexander Michael PROKUP, Peachtree Corners, GA (US); Terri WASMOEN, Peachtree Corners, GA (US)

(73) Assignee: HYPERCELL TECHNOLOGIES, Peachtree Corners, GA (US)

(21) Appl. No.: 18/400,972

(22) Filed: Dec. 29, 2023

Related U.S. Application Data
(60) Provisional application No. 63/478,037, filed on Dec. 30, 2022.

(51) Int. Cl. C12Q 1/70; C12N 15/10; C12Q 1/24; C12Q 1/6806; C12Q 1/6834; C12Q 1/6844; C12Q 1/689; C12Q 1/6895; G01N 21/64

(52) U.S. Cl. CPC C12Q 1/70; C12N 15/1096; C12Q 1/24; C12Q 1/6806; C12Q 1/6834; C12Q 1/6844; C12Q 1/689; C12Q 1/6895; G01N 21/6486; C12Q 2600/158

(57) ABSTRACT

Disclosed are compositions, devices and methods for processing biological and environmental samples for rapid detection of a gene of interest in a biological sample and uses thereof.

### CROSS-REFERENCE TO RELATED APPLICATIONS

This application claims the benefit of priority to U.S. Provisional Application No. 63/478,037, filed on Dec. 30, 2022, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 16, 2024, is named “067715.001US.xml” and is 82,190 bytes in size. The sequence listing contained in this .XML file is part of the specification and is hereby incorporated by reference herein in its entirety.

### FIELD

This invention relates generally to compositions, devices and methods for the rapid detection of a gene of interest in samples potentially containing biological material and uses thereof.

### BACKGROUND

Over the last decade, the direct cost of infectious diseases in animals has been estimated at more than $20 billion and indirect losses at over $200 billion to affected economies as a whole (Barratt Alyson S., et al., Frontiers in Veterinary Science, DOI 10.3389 (2019)). The impact of infections from animals spreading to humans is also staggering: WHO estimated that 11 major food borne diseases every year affect 48.4 million people and cause 59,724 deaths annually resulting in 8.78 million Disability Adjusted Life Years (DALYs) (Torgerson, Paul R et al., PLoS medicine, 12:12 e1001920 (2015)).

Infectious diseases in animals are costly by reducing animal productivity and requiring a number of treatments. As an example, it is estimated that parasitic worms cost the European livestock industry more than €1.8 billion per year, with drug-resistance costing at least €38 million per year in production losses and treatment costs. Biological contamination in the food chain is the cause of foodborne diseases. According the USFDA Foodborne diseases affect 48 million people in the USA every year, resulting in 128,000 hospitalizations and 3,000 deaths. It is estimated that 60% of food poisoning happens in restaurants. These diseases cost $55 billion a year to the US economy (Kowitt, B., Fortune Magazine (2015)). The pharmaceutical industry could also bear high costs due to biological contamination as these example demonstrate: a contamination in Genzyme's manufacturing plant costs the company $300 m in lost revenue in addition to $175 m fine by the US Food and Drug Administration; after a major contamination, Johnson & Johnson had to refit its manufacturing plant costing more than $100 m, in addition to the recall and reaction from the market that cost the company $1.6 bn.

Contamination of foodborne pathogens in the food production environment causes huge economic losses, attributed to waste of raw food materials, and poses a significant public health issue that can weaken the agricultural manufacturing sector. According to a risk assessment analysis done between Apr. 8, 2020-Feb. 8, 2022, 30% of ground pork products in retail settings were found to be contaminated with Salmonella, creating a huge risk of food borne illness to end consumers (USDA, Federal Register Notice Docket No. FSIS-2019-0023202 (2020)). In addition, there is a growing demand for testing genetically modified organisms present in the food or feed samples to meet the appropriate national and international controls, performing independent verification to trade in confidence with countries specifying GMO-free products, and preventing cross-contamination throughout the supply chain. The global GMO testing market is expected to grow from $1.85 billion in 2021 to $2.08 billion in 2022 at a compound annual growth rate (CAGR) of 12.3%. (GMO Testing Global Market Report October 2022).

In the past several years, the development and application of molecular diagnostic techniques have initiated a revolution in diagnosing and monitoring infectious diseases. However, most of these techniques have been developed using expensive, sophisticated lab equipment (e.g., thermocyclers with laser detection devices for polymerase chain reaction (PCR) technology) and requiring highly skilled operators in a centralized laboratory, which is expensive and time consuming. While highly sensitive and specific, the available tests take too long to facilitate quick action to reduce the food chain contamination and quarantine animals to prevent spread. Because of the high cost of these tests, their routine use has been limited to testing only for high risk (e.g., deadly) or economically important pathogens. The tests are too slow and expensive to be widely implemented to test for pathogen contamination in multiple points of observation in the food chain, in complex manufacturing plants or in farm animal production facilities.

For example, there are no testing methods quick enough to detect Salmonella positive pigs upon arrival at the meat-packing plant. If a quick test were available, it would allow segregation of positive animals for end-of-the day processing to prevent cross-contamination of meat from healthy pigs. Due to testing costs and timing, detection of pathogen contamination of meats is limited to final product testing that is used to determine eligibility for sale. Test technology limitations, therefore, are driving a reactive rather than a proactive approach to prevention of contamination of foods.

The lack of fast and cost-effective methods to detect pathogen contamination in fresh fruits and vegetables is even more costly, because of the short shelf-life for many of these products. In addition, exports of food products between countries is often delayed by slow tests to detect genetically-modified or incorrectly labeled food products. Therefore, there is a need for rapid and cost-effective tests to detect genetic material, such as pathogen contamination, in foods. Across all phases of food production, early detection of pathogenic contamination through rapid diagnostic detection minimizes risks of human disease, and associated recalls that damage brand image and reduce profitability.

It should be noted that any manufacturing processes that rely on biological materials could benefit from the use of a rapid test for the detection of genes of interest. For example, pharmaceutical manufacturing could benefit from earlier detection of microbial contamination. Vaccine manufacturing could confirm product identity and freedom from adventitious agents at much earlier stages of production.

Loop-mediated isothermal amplification (LAMP) is a rapid signal amplification method for the detection of DNA or RNA targets. LAMP requires exposure of pathogenic DNA to the LAMP primers and enzymes to facilitate amplification. Typical methods involve the use of commercial DNA extraction kits to generate purified DNA samples from various pathogens and sample types. These kits typically require 10 or more steps and at least 1 hour to complete the nucleic acid purification process. Use of these kits incurs additional cost and processing time, and requires additional expensive equipment like centrifuges, vortex mixers, and pipettes.

Thus, the invention presented herein provides point-of-care diagnostic compositions, devices and methods for the amplification and detection of pathogenic nucleic acids in sample. The methods presented herein can be performed quickly, inexpensively, and accurately to reduce the risks of foodborne diseases, pharmaceutical products recall or to limit spread of disease to other animals and potentially humans

### SUMMARY

In accordance with the purpose(s) of this invention, as embodied and broadly described herein, this invention, in one aspect, relates to a point-of-care/point-of-contact (POC) diagnostic system for detecting a gene-of-interest comprising: a sample obtained from a material-of-interest; a strand displacement DNA polymerase enzyme; reagents, buffers, diluents, serums, enzyme co-factors, and dNTPs; a set of at least 5 primers specific to the gene-of-interest; a heating and reader device and; data management software; wherein the POC diagnostic system completes target gene amplification and detection within 90 minutes of sample collection; and wherein the sample is optionally processed in less than four steps using additives, but without the purification of nucleic acids.

The POC diagnostic system further comprises a magnetic stick or magnetic comb for processing the sample by binding nucleic acids released from the sample, wherein the magnetic stick or magnetic comb comprise a body and a magnet, and wherein the body is bound to the magnet on the distal end of the body. The magnetic stick or magnetic comb may be used in methods of diagnosing an infection within a subject, wherein the magnetic stick is used to attract magnetic beads within the biological sample of the subject. Additionally, the magnetic stick or magnetic comb may be used in methods of detecting contamination in a food source, wherein the magnetic stick is used to attract magnetic beads within the biological sample collected from a food source.

In another embodiment, the POC diagnostic system further comprises a reverse transcriptase enzyme that catalyzes RNA-DNA conversion at temperatures between 60-70° C.

In one other embodiment, the material-of-interest is obtained from humans, non-human animals, plants, food, and water, and wherein the sample obtained from the material-of-interest is selected from a group consisting of serum, plasma, feces, urine, blood, oral fluids, bile, milk, colostrum, nasal secretions, oral secretions, ocular secretions, and fluids from tissues derived human, animal, other multicellular, complex species at risk for microbial diseases, minced, ground, mashed or similarly processed meat, fruits, vegetables, fish, bottled beverages, other processed food products with a risk of contamination with microbes, environmental surface at risk from contamination by infected animals or foods, a sample of water used to washed animals, foods, or environmental surfaces, or an enriched culture derived from the aforementioned samples.

In one embodiment, the gene-of-interest may be a marker of genetic modification that can determine species of origin for specific genetically modified plants or animals that are used to produce human food. In another embodiment, the gene-of-interest may be a marker of a microbial pathogen selected from a group consisting of a virus, bacterium, archaea, fungus, parasite or a microbial pathogen carrying a gene(s) facilitating resistance to a chemotherapeutic agent or antimicrobial drug used for treatment of disease, wherein the microbial pathogen infects a host's tissues and causes disease a host mammal, animal, insect, or plant or is a risk to cause disease in humans from contamination of food harvested from the host. The virus may be selected from porcine reproductive and respiratory syndrome virus (PRRSv), swine influenza virus, or avian influenza virus and wherein the bacterium selected from Salmonella enterica and its subspecies and serovars, Listeria, and E. coli.

In another embodiment, the reagents and enzymes presented herein are dried in one location of a test tube and the primers are dried in a separate location of said test tube, and wherein the enzymes and primers are dried under vacuum at room temp for 4 h to 24 h or under in a dry heat oven set at 40° C. to 60° C. and incubated for up to 2 hours or by freeze-drying. The reagents may be modified to reduce salts after sample addition in order to achieve a concentration under 100-120 mM and wherein the buffers and diluents for use with samples are modified to ensure pH ˜8.8 with detergents such as Tween or Triton added to the high pH lysis buffer.

In yet another embodiment, the serum is heated to 70° C. to 80° C. for 3 to 10 minutes to release pathogen nucleic acids and inhibit nucleases or other biomolecules that may be present in complex samples, and wherein the serum sample is treated with a high pH lysis buffer comprising sodium hydroxide or potassium hydroxide at pH >11 to facilitate release of nucleic acids from a microbe and inhibit nucleases or other biomolecules that may be present in complex samples.

In other embodiment, the POC diagnostic system comprises primers optimized to avoid primer-dimer pairs, hairpin loops, and other secondary nucleic acid interactions that limit DNA binding and/or polymerase recognition, and wherein the primer sets are selected from a group consisting of: a set of primers having 99% sequence identity of SEQ ID NO: 1, 2, 3, 4, 5, 6, 7, 8, 9, 10, 11, 12, or the combination thereof targets a ORF7 genetic target of the PRRV genome; a set of primers having 99% sequences identity to SEQ ID NO: 13, 14, 15, 16, 17, 18, 19, 20, 21, 22, 23, 24, 25, 26, 27, 28, 29, 30, 31, 32, 33, 34, 35, 36, 38, 39, 40, 41, 42, 43, 44, 45, 46, 47, 48, 49, 50, 51, or the combination thereof targets a invA of the Salmonella genome and can amplify Salmonella enterica strains Berta, Typhimurium, 4,[5],12:i, agona, and Braenderup; a set of primers having 99% sequences identity to SEQ ID NO: 52, 53, 54, 55, 56, 57, 58 or the combination thereof targets a uidA gene of an E. coli genome; and a set of primers having 99% sequences identity to SEQ ID NO: 59, 60, 61, 62, 63, 64, or the combination thereof targets H5N1 influenza A.

In one embodiment, the body of the magnetic stick or the magnetic comb is comprised of a low-density polyethylene, high density polyethylene, polypropylene, polyethylene terephthalate, polystyrene, polyvinyl chloride, polyoxymethylene, acrylic (polymethyl methacrylate), polyurethane, polycarbonate, polytetrafluoroethylene, polyetherimide, polyether ether ketone, nylon (polyamide), bamboo, wood, or metal (iron, aluminum, zinc, copper, magnesium, and alloys thereof). In another embodiment, the magnet of the magnetic stick or magnetic comb is comprised of neodymium, iron-nitride, tetraenite, hematite, magnetite, maghemite, alnico, ferrite, samarium cobalt, magnetic rubber, ilmenite, ulvospinel or electromagnet, and wherein the magnet attracts magnetic beads within the biological sample.

In another embodiment, the heating block and reader device is a small and compact box that heats, incubates, and reads at least 10 target gene amplification reactions at once.

In another aspect, the invention relates to a method for detecting a gene of interest in a material-of-interest comprising: processing a sample for direct use in target gene amplification reactions; generating a primer set targeted towards a conserved gene target; drying reagents onto a test tube, wherein reaction reagents, DNA strand-displacing polymerase, and reverse transcriptase are dried at one location of a test tube and primers are dried at a second location of said test tube by heat or vacuum; collecting a sample from the material-of-interest; diluting the sample in buffers to achieve appropriate salt concentration and pH; heating the sample to release pathogen nucleic acids and inhibit nucleases that may be present in the sample; resuspending the dried reagents in the test tube using the treated sample; inserting the test tube containing the treated sample into a heating and reader device capable of heating the sample at 65° C. and measuring fluorescence; amplifying the DNA within 90 minutes; and analyzing the measured fluorescence to detect the presence of the amplified DNA in the sample; wherein the method is performed using 1 sample, 1 tube and is completed within 90 minutes.

In one embodiment, the processing a sample for direct use in target gene amplification reactions comprising: a) lysing the sample to release nucleic acids from microbe; b) binding the nucleic acids to silica-coated magnetic beads; c) incubating the nucleic acid bound silica-coated magnetic beads with a magnetic stick to allow the binding of the silica-coated magnetic beads to the magnetic stick; d) washing the beads on the magnetic stick to remove contaminants; e) eluting of the nucleic acids from the beads bound to the magnetic stick into a buffer compatible with target gene amplification; f) transferring the eluted nucleic acids to a tube containing dried target gene amplification reagents for DNA/RNA amplification; wherein lysis of the sample and binding of the released nucleic acid to silica-coated beads are completed in a single step; and the sample is incubated with a lysis buffer combined with silica-coated beads and the sample matrix for 5 minutes at room temperature.

The lysis of the sample and binding of the released nucleic acid to silica-coated beads are completed in a single step. The sample is incubated with a lysis buffer combined with silica-coated beads and the sample matrix for 5 minutes at room temperature. In some embodiments, the lysis buffer contains chaotropic agents (urea, guanidinium salts), organic solvents (methanol, ethanol, propanol, butanol, acetone), enzymes (lysozyme, proteinase K), amino acids or polypeptides, buffering agents (Tris, HEPES, carbonate, phosphate, glycine, tricine, bicine, PIPES), or detergents (SDS, SLES, CHAPS, CHAPSO, n-octyl-beta-D-glucopyranoside, Triton X-100, Triton X-114, polysorbate, CTAB). In other embodiment, the magnetic stick bound with the silica coated beads is dipped into a wash tube for 10 seconds to remove any residual components of the lysis buffer. The magnetic stick is then transferred to an elution solution for 5 minutes, wherein the nucleic acids are released from the silica-coated beads. In some embodiments, the sample includes serum, plasma, feces, urine, blood, oral fluids, bile, milk, colostrum, nasal secretions, oral secretions, ocular secretions, and fluids from tissues derived human, animal, and other multicellular complex species at risk for microbial diseases; minced, ground, mashed or similarly processed meat, fruits, vegetables, fish, bottled beverages; other processed food products with a risk of contamination with microbes; environmental surfaces at risk from contamination by infected animals or foods; a sample of water used to washed animals, foods, or environmental surfaces; or an enriched culture derived from the aforementioned samples.

In one embodiment, the processing of the sample may further comprise uncoating a virus with a high pH buffer.

In another embodiment, the processing of the sample my further comprise a rapid lysis method for the direct addition of milk to target gene amplification reactions comprising; lysing a milk-containing sample by heat; adding the lysed sample to target gene amplification reagents in a PCR tube; adding polyaspartic acid to the sample in the PCR tube; and running a target gene amplification reaction; wherein the milk is whole milk, reduced fat milk, skim milk, buttermilk, powdered milk, condensed milk, or evaporated milk.

In an additional embodiment, the processing of the sample may further comprise a sample enrichment method comprising: binding a sample to a positively charged anion resin; separating the ion exchange resin by centrifugation, filtration, or magnetic force if bound to magnetic beads; incubating the resin and bound sample in a lysis buffer to concentrate and extract DNA into a smaller volume; adding the extracted DNA or RNA to target gene amplification reagents in a PCR tube; and running a target gene amplification reaction. The exchange resin is a positively charged anion exchange resin wherein the anion exchange resin is incubated with a negatively charged sample. The exchange resin may be a negatively charged cation exchange resin wherein the cation exchange resin is incubated with a positively charged sample. The anion exchange resin functionality may be a weakly basic anion (primary, secondary, and tertiary amines like dimethylaminopropyl, polyethyleneimine, or diethylaminoethyl) or a strongly basic anion (quaternary amines like diethyldialkyl ammonium chloride or alkyldialkyl ammonium chloride). In other embodiments, the cation exchange resin functionality is a weakly acidic cation (carboxylic acids) or a strongly acidic cation (sulfonic groups).

In yet another embodiment, the processing of the sample may further comprise a method of concentrating bacterial from dilute samples comprising: loading a sample into a first syringe pre-connected to a first syringe filter; depressing a plunger on the first syringe to force the liquid through the syringe, thereby trapping the sample on the syringe filter; transferring the first syringe filter with the trapped sample to a second syringe containing a smaller volume of lysis buffer; depressing the plunger on the second syringe to pass the lysis buffer through the first syringe filter, thereby passing the eluted sample genomic DNA through the filter; and adding the eluted genomic DNA or RNA to a target gene amplification reaction for pathogen detection; wherein the syringe filter has a 0.45, 0.22, or 0.1 filter comprising materials selected from a group consisting of nylon, polyethersulfone, cellulose acetate, regenerated cellulose, polypropylene, glass fiber, ceramic, metal, and wood; and wherein the method is completed in 5 minutes or less.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate several embodiment(s) of the invention and together with the description, serve to explain the principles of the invention.

FIG. 1 is illustrative of a prototype instrument developed for heating the LAMP reactions and detection of fluorescent output from amplification.

FIG. 2 shows the internal components of the prototype instrument developed for heating the target gene amplification P reactions and detection of fluorescent output from amplification.

FIG. 3 is illustrative of an apparatus for heating target gene amplification reactions and detecting fluorescence.

FIG. 4 is illustrative of the optical excitation source of the apparatus for heating target gene amplification reactions and detecting fluorescence, comprising a laser or a light emitting diode or some other light source.

FIGS. 5-8 are fluorescence intensity curves showing target gene amplification using reagents dried under vacuum and stabilized by 5% sucrose (FIG. 5), 8% trehalose (FIG. 6), 0.9% dextran (FIG. 7), and 0.9% dextran plus 2.5% glycerol (FIG. 8). Quantity of DNA amplified (proportional to fluorescence intensity) and time required for a positive reaction are shown for various samples. A synthetic DNA sequence was added as a positive control (red line). A negative control was included that did not contain any nucleic acid sequences (black line). RNA virus in serum was uncoated by heat at 75° C. for 5 minutes prior to addition to LAMP reaction (blue line).

FIGS. 9-10 are fluorescence intensity curves showing target gene amplification using reagents dried using heat and stabilized by 5% sucrose (FIG. 9) and 8% trehalose (FIG. 10). Quantity of DNA amplified (proportional to fluorescence intensity) and time required for a positive reaction are shown for various samples. A synthetic DNA sequence was added as a positive control (red line). A negative control was included that did not contain any nucleic acid sequences (black line). RNA virus in serum was uncoated by heat at 75° C. for 5 minutes prior to addition to target gene amplification reaction (blue line).

FIG. 11 is a fluorescence intensity curve showing the effect of a low temperature lysis method for PRRSV in serum (75° C. for 5 minutes). Quantity of DNA amplified (proportional to fluorescence intensity) and time required for a positive reaction are shown for various samples. Synthetic DNA was added as a positive control (red lines). A negative control was included that did not contain any nucleic acid sequences (black lines). The amplification of viral RNA prepared by incubation at 75° C. for 5 minutes (blue lines) was rapid.

FIGS. 12-14 are fluorescence intensity curves showing target gene amplification using pH 11.5 buffer (FIG. 12), pH 11.5 Buffer plus Tween-20 (FIG. 13), and pH 11.5 Buffer plus Tween-20 and Triton X-100 (FIG. 14) to release viral nucleic acids. Quantity of DNA amplified (proportional to fluorescence intensity) and time required for a positive reaction are shown for various samples. A synthetic DNA sequence was added as a positive control (red line). A negative control was included that did not contain any nucleic acid sequences (black line). RNA virus in serum was uncoated by heat at 75° C. for 5 minutes prior to addition to target gene amplification reaction (blue line).

FIG. 15 is a fluorescence intensity graph showing the amplification of PRRSV RNA processed by a pH lysis sample preparation method. A neutralization buffer was added to adjust the pH closer to 8.8 before amplification. Target gene amplification reactions were assembled from viral RNA prepared by the magnetic stick method (blue lines). A negative control was included that contained water instead of a nucleic acid target (black lines). The pH lysis method enabled rapid detection of viral RNA.

FIGS. 16-17 are fluorescence intensity curves showing the impact of varying concentrations of target gene amplification primers on RNA virus amplification (FIG. 16) and DNA gene amplification in the presence of 25% serum (FIG. 17). In FIG. 16, uncoated PRRS virus was added to Primer Mix 1 tested at dilutions ranging from 2.0× to 0.2× in a standard target gene amplification reaction. In FIG. 17, synthetic DNA was added to Primer Mix 1 tested at dilutions ranging from 2.0× to 0.1× in a standard target gene amplification reaction. The quantity of DNA amplified (proportional to fluorescence intensity) and time required for a positive reaction are shown for various primer concentrations.

FIG. 18 is illustrative of the commercial workflow for the preparation of nucleic acids from various sample types by the pH lysis method. Step 1 the sample is directly added to a tube containing high pH lysis buffer (Tube L) (a 1:4 dilution is used for serum, but is optimized for each sample type). Step 2 allows for viral uncoating (lysis) to occur during a 5-minute incubation at room temperature. Step 3 transfers a set amount of neutralization buffer (from tube N) to the sample tube. Step 4. The neutralized, uncoated viral sample (containing released genomic nucleic acids) is transferred to a tube containing dried target gene amplification reagents and primers, which is placed in the test device for incubation and reading.

FIGS. 19-22 are fluorescence intensity curves showing the amplification of Salmonella strains/serovars using invA target gene amplification primer sets 1 (FIG. 19), primer set 2 (FIG. 20), primer set 3 (FIG. 21) and primer set 4 (FIG. 22). Primer sets 1, 3, and 4 were tested with a mixture of genomic DNA from the Salmonella serovars agona, Berta, Braenderup, Typhimurium and 4,[5],12:i:- (blue lines). Primer set 2 was tested with Salmonella serovars agona, Berta, Braenderup, Typhimurium and 4,[5],12:i:- individually (blue lines). All primer sets included synthetic DNA positive control (red lines) and a negative control that did not include any nucleic acid (black lines). The quantity of DNA amplified (proportional to fluorescence intensity) and time required for a positive reaction are shown for various samples. Salmonella genomic DNA (gDNA) was purified prior to addition to target gene amplification reaction.

FIG. 23 is an illustration of a magnetic stick (15), wherein the body (16) of the magnetic stick is connected to a neck region (17) that is connected to a connector region (18). The connector region is then connected to a magnet (19) that attracts magnetic beads within the sample. FIG. 24 is illustrative of a magnetic stick (20), wherein the body (21) of the magnetic stick is directly connected to a magnet (19). Example dimensions are provided. The body of the magnetic stick can be longer or shorter (e.g., 140 mm) to provide enough length to utilize by an end-user, depending on the size of tubes used for the sample preparation process.

FIGS. 25-27 are fluorescence intensity graphs showing the amplification of genomic DNA from Salmonella in fecal samples using the magnetic beads and stick method. Three separate lysis buffers were used with FIG. 25 showing the results with buffer set 1, FIG. 26 showing buffer set 2, and FIG. 27 showing buffer set 3. Target gene amplification reactions were assembled from DNA prepared by the magnetic stick method (blue lines). A negative control was included that contained water instead of a nucleic acid target (black lines). The magnetic bead and stick method enabled rapid detection of bacterial DNA.

FIGS. 28 and 29 are fluorescent intensity curve graphs showing the amplification of viral RNA from swine PRRS virus prepared by the magnetic bead and stick method using two different sample prep buffer formulations. FIG. 28 shows the amplification of PRRS virus using buffer set 1 and FIG. 29 shows the amplification of PRRS virus using buffer set 2. A negative control was also included, which contained no nucleic acid, viral target, or bacterial target (black lines).

FIG. 30 is a fluorescent intensity curve graph showing the amplification of bacterial DNA from Salmonella Typhimurium prepared by the magnetic bead and stick method. The bacterial sample was collected by swab from a stainless-steel surface. A negative control was also included, which was a swab from a clean surface that contained no bacterial target (black lines). Salmonella collected from an environmental surface was processed by the magnetic bead and stick method to obtain genomic DNA. This process enabled rapid amplification in a target gene amplification assay.

FIGS. 31 and 32 are diagrams illustrating the commercial workflow for the food safety applications. FIG. 31 shows the workflow for the preparation of nucleic acids from various sample types using the magnetic stick and beads method. FIG. 32 shows the workflow for the parallel preparation of nucleic acids from multiple samples using the multiple magnetic sticks configured on a comb. The comb configuration spaces multiple magnetic sticks on one device that can simultaneously capture, wash, and elute magnetic beads from multiple sample tubes at once. For both figures, MS designates the magnetic stick, L is under the tube containing lysis buffer and magnetic beads, W is under the tube containing wash buffer, and E is under the tube containing elution buffer. Tubes from left to right are in the same order. The final step is transfer of eluted nucleic acids to the target gene amplification reaction tube (containing dried reagents and primers) that is inserted into the target gene amplification instrument for incubation and reading results.

FIGS. 33-35 are illustrations of a magnetic comb (22) for processing multiple samples. FIG. 33 shows the magnetic comb (22) having a spine (23) from which multiple magnetic sticks (24) protrude. Each of the multiple magnetic sticks has a magnet (19) on the distal end for processing samples. FIG. 34 is a front view of the magnetic comb (22) showing that the multiple magnetic sticks are connected by a 15 mm spacer (25). FIG. 35 is a side view of the magnetic comb (22).

FIG. 36 is a fluorescent intensity curve graph showing the detection of Escherichia coli (E. coli) in whole milk samples without PLD10 reagent using the direct sample addition method (blue lines). A negative control was also included, which contained no nucleic acid or bacterial target (black lines), which showed unexpected amplification within ˜45 minutes.

FIG. 37 is a fluorescent intensity curve graph showing the detection of E. coli in whole milk samples with added PLD10 reagent (direct sample addition method) (blue lines). A negative control was also included, which contained no nucleic acid or bacterial target (black lines).

FIG. 38 is a fluorescent intensity curve graph showing the capture and concentration of E. coli using anion exchange resin for direct addition to a target gene amplification reaction. A negative control was also included, which contained no nucleic acid or bacterial target (black lines).

FIG. 39 is a fluorescent intensity curve graph showing the concentration of bacteria from dilute solutions using a syringe filter. E. coli diluted in water to various concentrations was captured and concentrated in a syringe filter and eluted from the filter using a lysis buffer. The resulting eluate from a sample containing E. coli was directly added to a target gene amplification reaction. The total E. coli cfu filtered was 10,000 cfu (red lines), 1,000 cfu (green lines), and 100 cfu (blue lines). A negative control was also included, which contained no nucleic acid or bacterial target in the filtered solution (black lines). The syringe filter enabled rapid capture, concentration, and extraction of bacterial DNA for use in a target gene amplification reaction.

FIG. 40 is a fluorescent intensity curve graph showing the detection of E. coli from a swab of a contaminated lettuce leaf. The swab was eluted in water that was heated to 95° C. for 10 minutes before transfer to a target gene amplification reaction tube. The rapid amplification of E. coli from a swab collected from a contaminated leaf (blue lines), but not from a swab collected from clean lettuce (black line, negative control) confirms the specificity of this rapid method to screen fruits and vegetables for bacterial contamination.

FIG. 41 is a fluorescent intensity curve graph showing the detection of E. coli from a swab of a sample of peanut butter spiked with bacteria. The rapid amplification of E. coli from a swab collected from a contaminated peanut butter (blue lines), but not from a swab collected from peanut butter not intentionally contaminated (black line, negative control) confirms the specificity of this rapid method to screen a complex food matrix for bacterial contamination.

FIG. 42 is a fluorescent intensity curve graph showing the detection of highly pathogenic avian influenza virus using the target gene amplification assay. The rapid amplification of avian influenza from genomic DNA from a bird sample previously confirmed positive by classical qPCR (blue lines), but not a negative control (black line) confirms the specificity of this rapid amplification method to detect avian influenza viruses.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

The terms “patient”, “individual”, “subject”, and “animal” are used interchangeably herein and refer to mammals, including, without limitation, human and non-human veterinary animals (e.g., cats, dogs, cows, horses, sheep, pigs, etc.) and experimental animal models. The term “host” is used to refer to an animal or plant that is affected by a gene-of-interest, wherein the gene-of-interest is a marker of genetic modification that can determine species of origin for specific genetically-modified plants or animals that are used to produce human food or a marker of a microbial pathogen.

As used herein, the term “sample” refers to samples obtained from materials-of-interest. The term “materials-of-interest” refers to materials derived from human and non-human animals, plants, food, water, the environment and the like. Examples of the materials-of-interests are not limited to the examples provided below. The materials—of interest may be biological samples obtained from human and non-human animals such as tissue, serum, plasma, blood, feces, bile, nasal secretions, oral secretions, ocular secretions, milk, urine, whole eggs, egg yolks, egg whites, fluids from tissues derived human, animal, or other multicellular complex species at risk for microbial diseases. The materials—of interest may be food samples such as food ingredients from animal, plant, or insect origin, including but not limited to spices, additives, preservatives, food in progress, food components, finished products, sauces, fresh foods, frozen foods, fish, meat and processed meat products (ground or similarly processed), packaged beverages (in bottles, cartons, plastics etc.,) alcoholic beverages including sprits, beer, and wine, or other processed food products with a risk of contamination with microbes. The material of interest may be derived from plant products derived from whole plants. plant leaves, bark, and plant products such as fruits, vegetables, and grains. The materials—of interest may also be from water sources including but not limited to water run-offs, sewers, drinking water for humans, animals and plants, cleaning, and sanitation water, running water, retained water, water used in cleaning vegetation, and water used for cleaning meat products. The sample may be collected from an environmental surface at risk from contamination by infected animals or foods; or a sample of water used to wash animals, foods, or environmental surfaces. In some cases, the sample is taken from an enriched culture, wherein the enriched culture is derived from the aforementioned samples.

As used herein, the term “processing fluids” is defined as fluids derived from the tissues of a subject. In one embodiment, fluid is derived from testicle and tail tissues removed from the subject. In another embodiment, the subjects may be young piglets (swine).

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As used herein, the term “target gene amplification” refers to methods of amplifying and detecting a gene-of-interest using the point of care (POC) diagnostic system disclosed herein, also referred to as the “target gene amplification POC diagnostic system” or “target gene amplification POC diagnostic method”. The methods disclosed herein complete target gene amplification and detection within 90 minutes of sample collection. In the target gene amplification POC diagnostic system provided herein, target gene amplification methods such as loop-mediated isothermal amplification (LAMP), a nucleic acid-based technology, are used to selectively amplify a target DNA sequence using a set of up to six target gene amplification primers, recognizing six to eight regions of the target DNA sequence—hence a high specificity, and strand displacement polymerase under isothermal conditions. Reverse transcriptase is added to reactions where RNA is the nucleic acid in the test sample. Other amplification Methods for gene amplification include polymerase chain reaction (PCR; including but not limited to, real time-PCR and quantitative-PCR (qPCR)), ligase chain reaction (LCR), and transcription isothermal techniques such as transcription mediated amplification (TMA) or self-sustained sequence replication (3SR), nucleic acid sequence-based amplification (NASBA), signal-mediated amplification of RNA technology (SMART), strand displacement amplification (SDA), rolling circle amplification (RCA), loop-mediated isothermal amplification of DNA (LAMP), isothermal multiple displacement amplification (IMDA), helicase-dependent amplification (HDA), single primer isothermal amplification (SPIA), and circular helicase-dependent amplification (cHDA). mediated amplification (TCA), See, e.g., U.S. Pat. Nos. 4,683,195; 4,629,689; 5,427,930; 5,339,491; and 5,409,818. However, these technologies are limited by the number of multiple reagents with varying stability for such amplification as well as a reliance on expensive equipment.

As used herein, polymerase chain reaction (PCR) refers to an enzymatic nucleic acid amplification process that involves multiple cycles of denaturing template nucleic acid, annealing primers, and synthesizing a nucleic acid strand complimentary to the template strand. Each cycle will involve raising and lowering the reaction temperature to provide the proper thermal environment for each step of the cycle. Denaturing template nucleic acid is usually accomplished using high temperature, while annealing primers requires a lower temperature. Synthesis of the nucleic acid complementary to the template strand will typically occur at a temperature between the temperatures used for denaturing and annealing.

Within the scope of the disclosed methods, “thermocycle” refers to an automated process of changing temperature at fixed time intervals during each cycle of an amplification reaction. Thermocycling is often used in PCR because the denaturing, annealing, and synthesizing steps typically are performed at different temperatures.

A key embodiment of this disclosure is the ability of the target gene amplification diagnostic method to amplify microbial nucleic acids in the presence of complex biological material without the need for complex, multi-step, high-level purification of the nucleic acids. One such embodiment uses the dilution of serum to a 25% concentration in water, followed by heating the serum to 75° C. for five minutes then mixing with dried target gene amplification diagnostic reagents containing a reduced salt content. In some embodiments, this combination of treatments facilitated amplification of viral nucleic acids without the need for further purification.

As used herein, the term “microbial pathogen” refers to a virus, bacterium, archaea, fungus, and/or parasite that infects the host's tissues and causes disease in a host mammal, animal, insect, or plant and/or is a risk to cause disease in humans from contamination of food harvested from the host. The microbial pathogen may also carry a gene(s) facilitating resistance to a chemotherapeutic agent or antimicrobial drug used for treatment of disease.

In another embodiment, the dilution of serum to a 25% concentration was performed in a buffer resulting in a pH >11. This high pH was designed to release nucleocapsid proteins by disrupting ionic bonds thus releasing viral nucleic acids. After a 5-minute incubation, the diluted serum was neutralized by addition of liquid Tris buffer or added to dried Tris buffer in the bottom of the reaction tube (to return the solution to a pH ˜8.8), and then mixed with diagnostic reagents dried to the side of the tube. These steps also facilitated amplification of viral nucleic acids without the need for further purification. In another embodiment, Tween or Triton detergents are included in the pH >11 buffer.

As used herein, the phrase “nucleic acid,” and “nucleic acid sequence,” are interchangeable and not intended to be limiting. “Nucleic acid” shall have the meaning known in the art and refers to DNA (e.g., genomic DNA, cDNA, or plasmid DNA), RNA (e.g., mRNA, tRNA, or rRNA), and PNA. It may be in a wide variety of forms, including, without limitation, double-stranded or single-stranded configurations, circular form, plasmids, relatively short oligonucleotides, peptide nucleic acids also called PNA's and the like. The nucleic acid may be genomic DNA, which can include an entire chromosome or a portion of a chromosome. The DNA may include coding (e.g., for coding mRNA, tRNA, and/or rRNA) and/or noncoding sequences (e.g., centromeres, telomeres, intergenic regions, introns, transposons, and/or microsatellite sequences). The nucleic acid may include any of the naturally occurring nucleotides as well as artificial or chemically modified nucleotides, mutated nucleotides, etc. The nucleic acid can include a non-nucleic acid component, e.g., peptides (as in PNA's), labels (radioactive isotopes or fluorescent markers), and the like.

As used herein, the term “primer” or “target gene amplification primer” may adopt its customary meaning as understood by one of skill in the art. When used with LAMP or target gene amplification applications, the “primer set” refers to the 5-6 primers necessary to bind and amplify a gene of interest. The “primer set” is typically composed of a forward and reverse primer that flank the gene of interest, a forward inner primer and backward inner primer that replicate a double handle bar DNA motif exponentially, and 1-2 loop primers that further amplify the double handle bar DNA motif.

Reagents for LAMP or target gene amplification are known (e.g., Bst polymerase, dNTPs, buffers etc.). Moreover, primer pairs are easily designed and identified by one of skill in the art using known sequence information. For example, reagent preparations for loop-mediated isothermal amplification and target gene amplification of nucleic acids comprises at least one polymerase enzyme, wherein the enzyme is capable of strand displacement, a target-specific primer set, and deoxynucleotide triphosphates (dNTPs). In some embodiments, the polymerase enzyme capable of strand displacement is Bst enzyme. If the target is RNA, the reagent preparation also includes a reverse transcriptase. In some embodiments, the base structure of the reverse transcriptase is AMV reverse transcriptase or Moloney murine leukemia virus reverse transcriptase.

As used herein, “amplifying” and “amplification” refers to a broad range of techniques for increasing polynucleotide sequences, either linearly or exponentially. Amplification methods may be performed isothermally such as Loop-mediated isothermal amplification (LAMP). In various embodiments, the term “amplification product” or “amplified product” includes products from any number of cycles of amplification reactions.

As used herein, “lysis” refers to any chemical treatment or physical process which releases genomic nucleic acids from within cells or virus particles. In the case of viruses, this refers to disruptions of capsid proteins and, in some cases lipid envelopes, that surround viral genomes. In the case of bacteria, fungi, and parasites it refers to the disruption of cell walls, plasma membranes, and, in some cases nuclear membranes plus proteins, that surround genomic DNA.

Porcine reproductive and respiratory syndrome virus (PRRSV) is a relatively recently recognized swine pathogen associated with porcine reproductive and respiratory syndrome (PRRS). PRRSV is a significant pathogen in the swine industry. PRRSV infections are common in the U.S. swine herds. Outbreaks of PRRS in England have led to cancellation of pig shows.

Influenza virus is an enveloped RNA virus that uses differing hemagglutinin (HA) and neuraminidase proteins to infect a wide range of animals. Birds can be infection with at least 15 different HA types (Alexander, D. J. (2000). A review of avian influenza in different bird species. Veterinary microbiology, 74(1-2), 3-13.). But the H5 and H7 highly pathogenic strains carried by migrating birds have become a recurring problem for the chicken and poultry industries leading to widespread death due to disease and depopulation control measures (Verhagen, J. H., Fouchier, R. A., & Lewis, N. (2021), Viruses, 13(2), 212.). The H3N8 influenza viruses are most noted for causing disease in dogs and horses. The H1, H2, and H3 strains can cause severe disease in pigs and humans. Monitoring and control of influenza virus in swine is an area of focus because of economic losses associated with outbreaks, but more importantly because pigs can be infected with both avian and mammalian strains of influenza. Multiple human influenza pandemics had their origin traced to pigs, due to coinfection with and mixing of genes from avian and mammalian viruses in these animals (Trovio, N. S., & Nelson, M. I. (2020), PLoS pathogens, 16(3), e1008259.). Rapid diagnosis of influenza viruses at the farm could be an important tool to avert another pandemic.

Salmonella as used herein refers to a genus of rod-shaped, predominantly motile, enterobacteria. It can be found in animal, human, and non-living habitats.

As used herein, “Serovar” or “Serotype” is the short form of referring to the serological variants of bacteria, and is a way to distinguish between distinct types of bacteria that fall within a single species. The particular serovar of a strain refers to the individual classification of that bacteria within the species, as based upon cell membrane antigens.

Escherichia coli (E. coli) as used herein is a Gram-negative, rod-shaped, facultative anaerobic bacterium. Most E. coli strains harmlessly colonize the gastrointestinal tract of humans and animals as a normal flora. However, there are some strains that have evolved into pathogenic E. coli by acquiring virulence factors (e.g., toxin genes) through plasmids, transposons, bacteriophages, and/or pathogenicity islands. The designation “STEC” refers to Shiga Toxin-producing E. coli strains that cause a serious diarrheal disease in humans.

As used herein, “point-of-care”, “point-of-contact” or “POC” or “point of use” refers to a location at or near the location where the diagnostic system is used. A POC diagnostic system can be performed at the same place that the sample was collected. Examples as provided below are not intended to be limiting examples of locations where the POC diagnostic system may be used. In the case of human disease diagnostics, it refers to tests that do not require central laboratory facilities or highly trained technicians; and thus, can be done at home, in schools, at pharmacies, and many other locations. In the case of animal disease, it refers to tests that can be done on a farm, in a veterinarian's truck, in a veterinary clinic, in the owner's home, or any reasonable location close to the test animal. In the case of food safety, it refers to diagnostic tests that can be done in farm fields, in food storage facilities, in areas where food processing occurs, at abattoirs, at grocery stores, in restaurants, at import/export regulatory facilities, in homes, and many other places close to at-risk foods. In the case of plant pathology (fruits, vegetables, crops, forests and trees, ornamentals, gardens, golf courses, flowers, mushrooms, other plants), it refers to tests that can be done in the field or close to the field where plants are produced, in plant processing facilities, storage facilities, greenhouses, various transportation system for plants, grain and plant products as well as various places where plants are processed, stored, conditioned and shipped. In the case of water analysis, it refers to tests that can be done in places where analysis needs to be done on dormant or circulating water, cleaning water, run-offs, sewage, and any type of water system that can be contaminated with biological agents.

As used herein terms “ion-exchange” and “ion-exchange chromatography” refer to a chromatographic process in which an ionizable solute of interest (e.g., a protein of interest in a mixture) interacts with an oppositely charged ligand linked (e.g., by covalent attachment) to a solid phase ion exchange material under appropriate conditions of pH and conductivity, such that the solute of interest interacts non-specifically with the charged compound more or less than the solute impurities or contaminants in the mixture. The contaminating solutes in the mixture can be washed from a column of the ion exchange material or are bound to or excluded from the resin, faster or slower than the solute of interest. “Ion-exchange chromatography” specifically includes cation exchange (CEX), anion exchange (AEX), and mixed mode chromatography.

As used herein, an “anion exchange resin” refers to an insoluble matrix or solid support (e.g., beads) capable of having a surface ionization over a pH range of about 1 to about 14. In one embodiment, a strong anion exchange resin is a solid support having a surface coated with quaternized polyethyleneimine. An example of such a strong anionic exchange resin is the solid support of the CIMultus QA™ column. For example, the anion exchange resin may be a quaternary amine ion exchange resin. In a further embodiment, the anion exchange resin comprises trimethylamine and a support matrix comprising poly(glycidyl methacrylate-co-ethylene dimethacrylate). However, other suitable anion exchange resins may be selected.

As used herein, “cation exchange resin” or “CEX resin” refers to a solid phase which is negatively charged, and which has free cations for exchange with cations in an aqueous solution passed over or through the solid phase. Any negatively charged ligand attached to the solid phase suitable to form the cation exchange resin can be used, e.g., a carboxylate, sulfonate and others as described below. Commercially available cation exchange resins include, but are not limited to, for example, those having a sulfonate based group (e.g., MonoS, MiniS, Source 15S and 30S, SP Sepharose Fast Flow™, SP Sepharose High Performance from GE Healthcare, Toyopearl SP-650S and SP-650M from Tosoh, Macro-Prep High S from BioRad, Ceramic HyperD S, Trisacryl M and LS SP and Spherodex LS SP from Pall Technologies); a sulfoethyl based group (e.g., Fractogel SE, from EMD, Poros S-10 and S-20 from Applied Biosystems); a sulphopropyl based group (e.g., TSK Gel SP 5PW and SP-5PW-HR from Tosoh, Poros HS-20 and HS 50 from Applied Biosystems); a sulfoisobutyl based group (e.g., (Fractogel EMD S03 “from EMD); a sulfoxyethyl based group (e.g., SE52, SE53 and Express-Ion S from Whatman), a carboxymethyl based group (e.g., CM Sepharose Fast Flow from GE Healthcare, Hydrocell CM from Biochrom Labs Inc., Macro-Prep CM from BioRad, Ceramic HyperD CM, Trisacryl M CM, Trisacryl LS CM, from Pall Technologies, Matrx Cellufine C500 and C200 from Millipore, CM52, CM32, CM23 and Express-Ion C from Whatman, Toyopearl CM-650S, CM-650M and CM-650C from Tosoh); sulfonic and carboxylic acid based groups (e.g., BAKERBOND Carboxy-Sulfon from J.T. Baker); a carboxylic acid based group (e.g., WP CBX from J.T Baker, DOWEX MAC-3 from Dow Liquid Separations, Amberlite Weak Cation Exchangers, DOWEX Weak Cation Exchanger, and Diaion Weak Cation Exchangers from Sigma-Aldrich and Fractogel EMD COO— from EMD); a sulfonic acid based group (e. g., Hydrocell SP from Biochrom Labs Inc., DOWEX Fine Mesh Strong Acid Cation Resin from Dow Liquid Separations, UNOsphere S, WP Sulfonic from J. T. Baker, Sartobind S membrane from Sartorius, Amberlite Strong Cation Exchangers, DOWEX Strong Cation and Diaion Strong Cation Exchanger from Sigma-Aldrich); and a orthophosphate based group (e.g., PI 1 from Whatman).

### II. Quantifying Salmonella Contamination

Salmonella is a leading cause of foodborne illness, with 1.3 million cases of salmonellosis occurring annually in the U.S. (Bearson, S. M. D., Annu Rev Anim Biosci., 10:373-393 (2022)). Salmonella enterica is the type species and is further divided into six subspecies with S. enterica ssp. enterica as subspecies that includes over 2500 serovars. These serovars are ubiquitous in the environment and can colonize food producing animals and poultry as well as wild animals and birds without causing overt disease. Examples of Salmonella serovars include, but are not limited to, S. enterica serovar Typhimurium, S. enterica serovar choleraesuis, S. enterica serovar Heidelberg, S. enterica serovar Paratyphi, S. enterica serovar Dublin, S. enterica serovar derby, S. enterica serovar London, S. enterica serovar Enteritidis, S. enterica serovar arizonae, S. enterica serovar anatum, S. enterica serovar berta, S. enterica serovar 4,[5],12:i:-, S. enterica serovar agona, S. enterica serovar Braenderup, S. enterica serovar infantis, S. enterica serovar Putten, S. enterica serovar Johannesburg, S. enterica serovar Eko, S. enterica serovar Schwarzengrund, S. enterica serovar uganda, and S. enterica serovar Senftenberg.

Contamination of food products with Salmonella is not only a serious health issue, but also a significant economic impact to food producers with an annual cost of over $2 billion to the food industry (Magossi et al., 2019).

Contamination of food products with Salmonella is a recurring problem, causing at least 1 recall every year since 2010. According to a report from the FSIS for calendar year 2021, over a million pounds of food product was impacted from only 4 Salmonella-based food recalls. Pork products are especially susceptible to contamination, with over 30% comminuted pork and 9% of pork cuts confirmed to contain Salmonella (USDA Federal Register Notice Docket No. FSIS-2019-0023202 (2020)). Salmonella is found not only in finished food products but also at swine farms. A recent study from 2022 found that Salmonella was present in 11.3% of healthy pigs (Karabasanavar et al., Brazilian Journal of Microbiology, 53:1039-1049 (2022)). Simply segregating these infected pigs before harvest based on symptomatic visual cues is not possible, as these were all otherwise healthy and non-diarrheic. Instead, direct detection of Salmonella is required to determine if the bacterium is present in pigs before slaughter.

Salmonella is a major problem for poultry producers as well. Between 1998 and 2008, poultry accounted for 17.9% of foodborne illnesses in the United States, with Salmonella ser. Enteritidis and Typhimurium are responsible for 17.4% and 34% of poultry-related foodborne illnesses, respectively (Painter J. A., et al., Emerg. Infect. Dis. 2013; 19:407). An adequate diagnostic and disease prevention program is essential to a profitable commercial poultry operation.

Salmonella is shed in the feces of infected animals. Salmonella deposited in feces on soil can survive for long periods of time and can spread to adjacent areas through the blowing dust. The recent Salmonella contamination found in flour is believed to have been caused by wheat contamination by soil and dust from contaminated field (Magallanes López, A. M., & Simsek, S. (2021), Cereal Chemistry, 98(1), 17-30). Fecal contamination of ground water and drinking water can lead to Salmonella infection of people (Popa, G. L., & Papa, M. I. (2021), Germs, 11(1), 88). The recent outbreaks of Salmonella due to melons is likely due to contamination from soil or water that was contaminated by feces from Salmonella-infected animals.

### A. Pork Manufacturing and Lairage Pen Contamination

The pork manufacturing process begins with a shipment of pigs to the meat packing plant where they are held in lairage, a pre-harvest transient holding pen. These transient pre-harvest lairage pens is one area where Salmonella is spread amongst other members of the herd immediately prior to the food manufacturing process (Vieira-Pinto et al., International Journal of Food Microbiology, 110(1):77-84 (2006)). Along with potential amplification in lairage, subclinical pigs are harvested and contaminated trim meat is combined from multiple sources. This trim is then ground, potentially contaminating the meat from Salmonella-negative pigs.

Plants are required to test 5 times a month, and the focus of these diagnostics is on post-harvest final products. The current testing paradigm for pork manufacturers is proprietary and contained in their HACCP (Hazard Analysis and Critical Control Point) plans. But an example procedure, as described by the FSIS, includes a preliminary identification test followed by a culture-based selection for Salmonella and MALDI-based serotype identification (USDA, MLG 4 Appendix 2.06 (2021)). Overall, the process can take anywhere from about 16 hours to 6 days. For preliminary identification tests, the majority of the testing duration is due to a pre-enrichment culture step, which can take 15 hours or longer. Use of these tests for pre-harvest detection would be ineffective at responding to the short lairage holding durations, which can be as quick as 1 hour. The processing of beef also uses a lairage system, where the spread of both Salmonella and E. coli contamination is of concern.

### III. Target Gene Amplification

Provided herein POC diagnostic system for detecting a gene-of-interest, wherein the POC diagnostic system completes target gene amplification and detection within 90 minutes of sample collection. In the POC diagnostic system provided herein, target gene amplification methods such as loop-mediated isothermal amplification (LAMP), a nucleic acid-based technology, are used to selectively amplify a target DNA sequence using a set of up to six primers, recognizing six to eight regions of the target DNA sequence—hence a high specificity, and strand displacement polymerase under isothermal conditions. In certain embodiments, the target DNA is amplified using a set of at least 5 primers specific to the gene-of-interest. The auto-cycling reactions lead to accumulation of a large amount of the target DNA and other reaction by-products, such as magnesium pyrophosphate, that allow rapid detection using varied formats (Njiru, PLOS Neglected tropical Diseases, 6(6):e1572 (2012)).

LAMP is well known for its robust and highly sensitive and specific amplification of target DNA, which is achieved by utilizing the set of five to six primers. Moreover, LAMP excels through its isothermal and energy efficient amplification requirements, rendering it a prime candidate for low-cost diagnostics and analysis at the point of need. This technology fits with the recommendation of the WHO for a molecular test suitable for developing countries, and by extension for wider and more frequent usage in developed countries. The World Health Organization (WHO) recommends that an ideal diagnostic test suitable for developing countries should be Affordable, Sensitive, Specific, User-friendly (simple to perform in a few steps with minimal training), Robust and rapid (results available in 30 min), Equipment free, and Deliverable to the end user (ASSURED).

Following these guidelines, it is clear that the LAMP technology has many benefits over the current technologies to address the un-met needs as described above. The technology is affordable and does not require expensive thermal cycling devices that are necessary for qPCR. The nucleic acid amplification can be done at 60-70° C., using simple heating devices that do not require skilled operators. The technology is also sensitive, having the same sensitivity (limit of detection) as qPCR and improved specificity by using the series of 5-6 primers, instead of the two used by qPCR. The risk of false positive results in diminished by selection of specific genes that are unique to the pathogen of interest. The technology also provides the advantage of being easily to adapted to changes in the microbes of interest because new primers can be developed quickly. Thus, for pathogens with high levels of genetic variability, the LAMP test can be quickly modified to detect new strains. LAMP is also user-friendly, not needing complex equipment, having a reduced number of steps to prepare and process the samples, allowing simple reading of the results (positive results can be visualized by a color change, fluorescence generated after intercalation of a dye into DNA, or the presence of turbidity (cloudiness) that can be visualized with the naked eye). The technology is robust and is rather forgiving for sample purity because LAMP typically uses Bst polymerase, which is capable of polymerizing DNA strands in the presence of inhibitors. It is therefore well suited to perform in “dirty” environments: at the farm; in processing plants; on the manufacturing floor. Results are rapidly obtained in under one hour. The technology has the advantage of requiring low-cost equipment, with no need for a complex thermocycler. The only equipment is a combination of a heating block and a reader, which can be combined in a small and compact “box” with a footprint of less than 1 sq foot. Therefore, tests run in a small device (“box”) which is portable, light and rugged would be ideal for POC facilities (farms, manufacturing plants, etc.).

However, the current LAMP technology needs significant modifications before it will be useful in a farm or meat packing plant setting. In order to detect RNA viruses, a reverse transcriptase step is needed to convert viral RNA to a DNA template that works with the LAMP polymerase. Current industry standard reverse transcriptase enzymes have optimal activity at temperatures lower than used for LAMP amplifications. The need for incubation of test samples using multiple incubation temperatures can be prone to errors, if done by workers lacking scientific knowledge.

Under laboratory conditions, LAMP assays have been shown to have sensitivity that is similar to qPCR. However, this level of sensitivity has only been demonstrated when using samples where the microbial DNA has been purified and concentrated. Current DNA purification methods require specialized equipment and scientifically skilled operators, which will not be available in a farm setting.

Furthermore, LAMP equivalence to qPCR has only been demonstrated when using fluorescent readouts. Therefore, colorimetric or turbidimetric methods may not be sensitive enough. The current optical sensors used in qPCR instruments to read fluorescence are too expensive and complicated to use in a farm setting. Therefore, new optical sensors or modifications to colorimetric/turbidimetric readouts are needed.

Currently, LAMP enzymes and reaction components are sold as frozen reagents. Farms and other animal POC facilities do not have scientific grade freezers (no manual defrost cycles) available for storing these reagents. New formulations are needed to facilitate the storage of test kits at typical temperatures (65-75° F.) or wider ranges that may be common in farm settings.

The majority of research into LAMP assays has been done for human disease detection. In this case, the work was done in centralized labs with skilled technicians, so they could use purified DNA for LAMP assays. A test done on a farm or manufacturing plant needs to be much simpler. If possible, there should be no processing to isolate microbe nucleic acids. However, little is known about components in biological fluids that could inhibit LAMP reactions.

Preliminary studies testing for amplification of synthetic nucleic acids in serum showed that unknown factors in serum inhibited the amplification of DNA in LAMP assays. Serum is likely to be the cleanest sample that will be tested in a farm setting. If LAMP reagents are inhibited by serum, there is little chance that these assays will work on more complex samples. For example, a common method to collect saliva from pigs or cows is to hang nylon ropes in pens containing multiple animals. These ropes will contain saliva, but also may be contaminated with environmental materials like food debris, dust, and bedding. Nasal swabs from animals collected in a barn are also likely to be contaminated with dust and dander that is not common in human nasal swab samples. Fecal samples from farm animals are often collected by walking through manure in pens with plastic boot covers. Fecal samples collected from disposable boot covers are likely to have a complex mixture of feces, dirt, bedding, etc. that will complicate LAMP testing for pathogens best detected in feces. Simple methods to deal with these complex sample types will be needed to facilitate POC LAMP tests that are compatible with farm settings.

The current described methods for LAMP diagnostic require up to 14 individual steps, which, could impede its implementation at POC facilities (farms, manufacturing plants, etc.). It is important to find optimized processes to reduce the number of steps from the treatment of the sample to the reaction itself.

Most LAMP assay heating and reading devices have been developed for human health purposes and are still too expensive to be used with the necessary frequency at POC in production animal facilities, manufacturing plants or along the food chain.

LAMP has previously been adapted to the detection of Salmonella in over a hundred academic applications (Dehghani et al., Food Control, 121:107664 (2021); Ghorashi et al., Avian Pathology, 51(5):476-487 (2022); Ou et al., Palliat. Med, 10:6850-6858 (2021); Yang et al., Foodborne Pathogens and Disease, 15(6):309-331 (2018)). Overwhelmingly, these applications are on the post-harvest detection of Salmonella in food products and are not compatible with testing the pre-harvest samples such as from lairage pens.

In an example by Dehgahni et al., a LAMP assay was developed for the detection of Salmonella in chicken fecal samples (Dehghani et al., Food Control, 121:107664 (2021). The rapid test was completed in under 3 hours, did not incorporate an enrichment step, and detected a sensitivity down to 3-10 CFU/mL. However, the assay used a number of specialized materials that may be difficult to cost-effectively commercialize. Ou et al. used fecal samples in a LAMP assay without an enrichment step; however, their assay relied on purified genomic DNA (Ou et al., Palliat. Med, 10:6850-6858 (2021)). Ghorashi et al. also used purified genomic DNA from fecal samples in a Salmonella LAMP assay, but after a 22-hour enrichment step (Ghorashi et al., Avian Pathology, 51(5):476-487 (2022)).

Commercial diagnostic products have also applied LAMP technology to the detection of Salmonella in post-harvest food products. One example from 3M is the “Molecular Detection Assay 2—Salmonella” (Bird et al., Journal of AOAC International, 99(4):980-997 (2016)). This assay utilizes an upstream enrichment step, which significantly extends the overall duration of the diagnostic process to over 16 hours, and limits applicability of the assay in POC test settings. Operation of the device is also quite involved, requiring multiple pre-heating steps (30 minutes to pre-warm the heat block, 20 minutes to pre-warm the measurement device, 2 hours to warm lysis solution).

Another commercial LAMP kit for the detection of Salmonella in chicken fecal samples was validated and compared against real time PCR and culture methods (di Bella et al., Applied Sciences, 11(15):6669 (2021)). Though the assay boasted a 0.22 CFU/g limit of detection, the process involved a 22-hour pre-LAMP enrichment step.

Therefore, previous uses for LAMP to detect Salmonella on farms or in food samples have not resulted in tests meeting the requirements for POC according to ASSURED.

### A. Target Gene Amplification POC Diagnostic System for Viral Diagnosis in Animal Samples

Serum samples are routinely collected and analyzed for the diagnosis of pathogens in animals. Typical workflows utilizing serum samples include multi-step nucleic acid purification protocols performed in laboratory environments that require expensive and specialized instrumentation. But these protocols are incompatible with diagnostics for POC settings, such as a farm or manufacturing facility, where adoption of animal diagnostic products is driven by cost and ease of use by untrained personnel. Technologies like LAMP/reverse transcriptase (RT)-LAMP are readily adapted to these POC settings, requiring only a simple low-cost device for heating and assay readout.

However, initial investigation has revealed some challenges when using serum in an RT-LAMP reaction:
1. Poor amplification in 100% serum, as indicated by low fluorescence signal.
2. Virus in serum does not appear to uncoat (release the nucleic acids) sufficiently at 65° C. based on low amplification signals.
3. The poor amplification in 100% serum appears to be due to the pH of serum (pH 7), which is much lower than the optimum pH for LAMP polymerases (˜pH 8.8).
4. The poor amplification in 100% serum may also be due to the high salt content of serum (150 mM) when compared to the lower isotonic strength (<100 mM) preferred by LAMP enzymes.
5. The poor amplification in 100% serum may also be due to the presence of nucleases that destroy DNA/RNA and/or other undefined inhibitors of RT-LAMP reaction.

Additionally, LAMP reagents are commonly supplied in frozen form. The facilities where point of care LAMP assays will be needed will likely not be lab facilities, having readily available freezers. Therefore, a room temperature stable formulation of the enzymes and key components is needed.

To solve the challenges presented above, the target gene amplification POC diagnostic system provided herein includes a step to pre-dilute serum before addition to a target gene amplification reaction. The diluent used is formulated to ensure salt and pH compatibility with target gene amplification reactions. However, in some variations, chemical additives are included to enhance viral uncoating to release nucleic acids. Also provided herein are target gene amplification reagents that have also been dried to increase shelf-life and reduce steps (i.e., pre-dried into a reaction tube). Additionally, target gene amplification reagents enzymes are provided herein.

Also disclosed herein, are target gene amplification primers for use in the target gene amplification POC diagnostic system. Sequences of primer sets for the detection of PRRSV ORF7 genetic target are presented in Table 1. An alternative primer set for the detection of the PRRSV ORF7 genetic target is provided in Table 2. In one embodiment, the disclosure presents a set of primers for the detection of PRRSV having 85%, 90%, 95%, 99%, or 100% sequences identity to SEQ ID NO: 1, 2, 3, 4, 5, 6, 7, 8, 9, 10, 11, 12 or the combination thereof.

Sequences of primer sets 1-4 for the detection of Salmonella invA gene are provided in Table 4, and alternative primer set sequences for detection of the invA gene in Salmonella spp are provided in Table 5. In another embodiment, the disclosure presents a set of primers for the detection of the invA gene in Salmonella spp. having 85%, 90%, 95%, 99%, or 100% sequences identity to SEQ ID NO: 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or the combination thereof. The primer sequences presented herein target the Salmonella gene invA and can amplify Salmonella enterica strains Berta, Typhimurium, 4,[5],12:i, agona, and Braenderup.

Sequences of uidA primer set used to detect E. coli in a target gene amplification reaction are provided in Table 6 and an alternative primer set for the detection of the E. coli uidA genetic target is provided in Table 7. In other embodiments, the disclosure presents a set of primers for the detection of the E. coli uidA genetic target having 85%, 90%, 95%, 99%, or 100% sequences identity to SEQ ID NO: 52, 53, 54, 55, 56, 57, 58, or the combination thereof.

Sequences of the H5N1 influenza A primer set a target gene amplification reaction are provided in Table 8. In other embodiments, the disclosure presents a set of primers for the detection of the H5N1 influenza A genetic target having 85%, 90%, 95%, 99%, or 100% sequences identity to SEQ ID NO: 59, 60, 61, 62, 63, 64, or the combination thereof.

The target gene amplification POC diagnostic system disclosed herein also provides methods for the rapid uncoating of viral particles in serum. Uncoating viral particles is a crucial first processing step for any sample undergoing detection by reverse transcriptase (RT)-target gene amplification. Exposure of the RNA to a reverse transcriptase enzyme enables synthesis of a cDNA transcript, which can be directly amplified in a target gene amplification reaction. Conventional methods to uncoat viral particles typically involve high temperature conditions (e.g. 95° C. for 1 minute). However, these high temperatures are incompatible with serum samples. At high temperatures, the serum samples will gel and precipitate, preventing the volumetric transfer of serum to a target gene amplification reaction and reducing diffusion of viral RNA within the reaction solution. The high heat may also begin to damage the RNA template, which is counterproductive to the goal of diagnostic detection. These issues experienced when using high heat conditions can be overcome by using a lower temperature for slightly longer duration (e.g. 75° C. for 5 minutes). The lower temperature minimizes the gelling of serum components, while the longer duration still enables sufficient uncoating of the viral genome. By lowering the temperature closer to the stability range of some commonly used RT-LAMP enzymes (e.g. Bst polymerase, reverse transcriptase), a heat-based viral lysis step could be potentially integrated with RT-target gene amplification enzymes for a one-pot detection method.

The use of heat as a lysis method still requires additional heating devices and steps that may be inconvenient for POC settings and it may not work for all types of viruses. Instead, an improved viral uncoating method would be performed without additional hardware at room temperature, with cheap reagents, and in a short timeframe. Dilution of serum coupled with pH modification can accomplish viral uncoating at room temperature without the need for additional heating devices, within 5 minutes, and only uses a single cheap alkali reagent. The all-liquid format integrates well with serum samples, which can double as a dilution step. Initial investigation also revealed that a short 5 minute incubation at the high pH appears to minimize the effect of nucleases and target gene amplification inhibitors present in the serum sample. Consolidation of the pH-based lysis with the final target gene amplification reaction can be easily performed by ensuring a high capacity buffer is included in the reagent mixture.

1. pH-Based Viral Lysis with Dried RT-Target Gene Amplification Reagents

Provided herein is a pH-based viral lysis method for the detection of viruses in serum. A sample of serum is diluted to less than or equal to 50% in a lysis buffer composed of aqueous sodium hydroxide. After incubation at room temperature for 5 minutes, the pH can be adjusted lower by addition of Tris or another buffer. Then the uncoated virus solution is mixed with target gene amplification reagents dried in a PCR tube. Alternatively, the buffering agent used to neutralize the alkaline serum can be dried in the target gene amplification reaction tube along with the other reaction reagents. After resuspension of the RT-target gene amplification reagents and primers, the reaction may be incubated at 65° C. to initiate amplification and fluorescence generated by the reaction will be measured.

### 2. Improved Reverse Transcription

Many viruses have RNA genomes. To be amplified by LAMP the RNA must first be transcribed to DNA. A commonly used LAMP polymerase, Bst, can convert RNA to a DNA copy (called cDNA), but it is very inefficient. Therefore, most RT-LAMP assays add a reverse transcriptase enzyme to make the RNA to DNA conversion faster. Reverse transcription (RT) enzymes used for sequence analysis have RNase H activity that degrades the RNA strand after reverse transcription. To ensure more copies are made from the viral genome for LAMP, an RT enzyme without RNase H might improve performance. Classic reverse transcriptase enzymes function optimally at temperatures lower than the optimal LAMP reaction temperature of 65° C. (e.g. from 37 to 45° C.). In order to increase sensitivity, the amount of RNA converted to cDNA is critical. Therefore, it makes sense to screen RT enzymes to find the most efficient enzymes to use for our desired testing conditions.

The following alternative RT enzymes have been identified that have improved enzymatic activity at 65° C. and/or no detectable RNase H activity: RapiDxFire (Lucigen), WarmStart Luna RT (NEB), Tth (Bioron), Ultrascript 2.0 (PCR Biosystems), and Codex HiTemp (Codexis) that may be incorporated into specific target gene amplification assays for improved performance.

### 3. Inhibition of Nucleases and Other Biomolecules

Nucleases are enzymes that breakdown RNA or DNA and are commonly found in serum and other body fluids. If present in test samples, these enzymes can destroy the target nucleic acids and/or the DNA that has been amplified by a LAMP or target gene amplification reaction. A necessary cofactor for nucleases, like DNase I, is calcium. Chemicals that chelate calcium can greatly reduce nuclease and other biomolecules activity in biological samples. A list of commonly used laboratory calcium-specific calcium chelators include: EGTA, BAPTA, EDTA (though this binds magnesium, which is needed for LAMP or target gene amplification), and citrate (though this binds magnesium, which is needed for LAMP). These chelators may be added to sera to improve the performance for specific target gene amplification assays as needed.

### B. Rapid Detection of Salmonella in Fecal Samples

A major source of Salmonella contamination within retail pork and beef products has been traced to the pre-harvest lairage pens used by meat packing plants to hold animals before slaughter. Just prior to entering these lairage pens, the animals are transported through loading bays, where fecal samples are deposited by the animals. These fecal samples can be easily acquired and investigated for the presence of Salmonella. If the bacteria is detected from the loading bay fecal samples, the pigs or cattle can be properly managed by the meat packers before release from the lairage pens.

Due to the temporary nature of lairage pens, all of the sample processing and diagnostic detection must be streamlined to be completed in about 1 hour. This ensures that any animals entering lairage pens can be properly diagnosed and controlled before release into the meat packing plant. Otherwise, a single undiagnosed animal with a Salmonella or other infection can contaminate any subsequent products processed in the facility.

In order to detect Salmonella from fecal samples from pre-harvest environments the following methods are required: novel collection devices, extraction of Salmonella from the collection device, rapid enrichment for Salmonella, lysing of the bacteria, and detection of the amplified genomic DNA in a LAMP assay. Novel collection devices, such as dusters, filter paper, cloths, or sponges, allow quick and easy collection from pre-harvest environments. Sample processing requires special buffer formulations designed to efficiently and quickly separate Salmonella from the collection devices. A key aspect of the invention to enable the rapid detection of Salmonella in fecal samples requires suspension of fecal samples in a lysis buffer that will release bacterial DNA for capture by silica-coated magnetic beads. The purpose of the beads is to directly bind and capture Salmonella genomic DNA and facilitate washing away fecal inhibitors of amplification. After washing and elution of DNA from the beads, the concentrated DNA can be added to dried target gene amplification reagents for rapid isothermal amplification and detection by fluorescence.

Current diagnostic assays used for the detection of Salmonella incorporate culture-based methods, with incubation times upwards of 10 hours. Additionally, these assays typically employ complex nucleic acid purification processes. These methods are suitable for final products, where longer assay durations are more easily tolerated for infrequent testing. The method developed to detect Salmonella in feces from pre-harvest lairage environments provides of results in 1 hour, which is not possible using culture-based methods. This more affordable diagnostic method which uses fewer steps, fewer reagents, and less time will also be a perfect tool to monitor carcass and meat samples during processing as a way to manage risk. More rapid testing will allow plants to divert meats with higher levels of Salmonella to pre-cooked products. This test is also ideal for monitoring Salmonella on environmental surfaces and equipment that contact contaminated meat. Monitoring before and after cleaning and disinfection will provide rapid information that processes are performed correctly to reduce risk. Overall, the devices and methods provided herein offer a simplified diagnostic process that will enable rapid and more frequent testing for Salmonella contamination and an important new tool to prevent outbreaks.

### C. Instrumentation for Performing Target Gene Amplification Assays in a POC Setting

The target gene amplification assay will need to be heated and signal generated by the assay will need to be measured. In another aspect, the invention provides an apparatus for heating target gene amplification reactions and detecting fluorescence or other changes in the optical properties of the solutions such as color or turbidity. In an embodiment, a fluorescing dye may be excited by a light source, for instance by light emitting diodes LEDs, tuned to be absorbed by the fluorescent dye. The dye may absorb the light from the light source and emit light at a different wavelength. In an embodiment, the dye may be SYBR Green I from Sigma Aldrich. The reader device may detect the light emitted by the dye. An exemplary reader device may comprise a photodetector in addition to filters or optical components to collect the emitted light and to exclude light of other wavelengths. In the example of SYBR Green I, the dye absorbs light at a wavelength of approximately 480 nm (blue) and emits at a wavelength of approximately 520 nm (green). In this example, the excitation light source may comprise an LED tuned to emit light at approximately 480 nm and the reader may be a photodetector with optical components designed to detect light of approximately 520 nm. The reader device may also comprise an electronic circuit or circuits to excite the fluorescence and to collect the emitted light while recording the results over time (e.g., the intensity of the emitted light as a function of time). The reader may be incorporated into the heating element or the heating element may be incorporated into the reader device. In an example, the heating element may comprise a block of material formed to surround the test tube in order more easily sustain a uniform temperature for the sample.

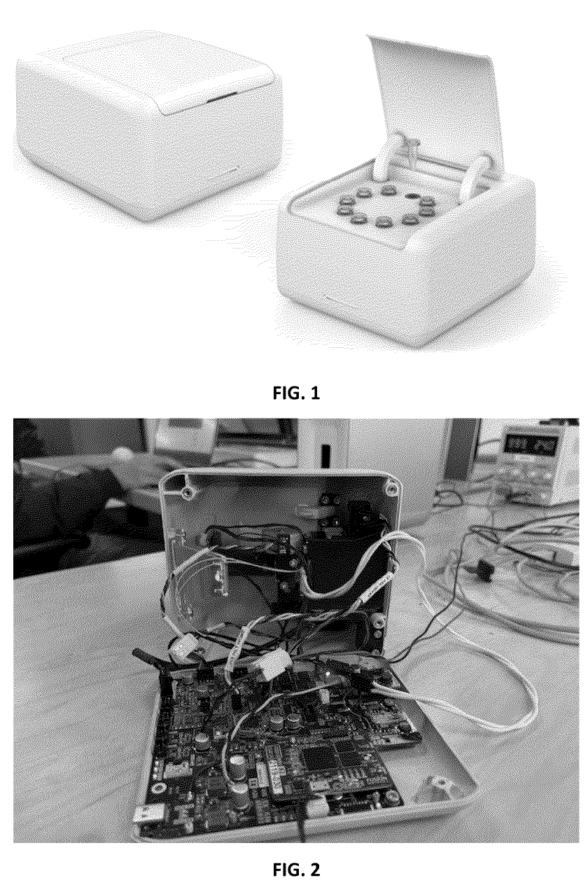

FIG. 1 shows the prototype instrument developed for heating the target gene amplification reactions and detection of fluorescent output from amplification. The prototype with a capability of reading at least 8 samples has a small footprint (˜6×8 inches) that is easily portable for POC applications. It can be powered from a standard electrical outlet or battery.

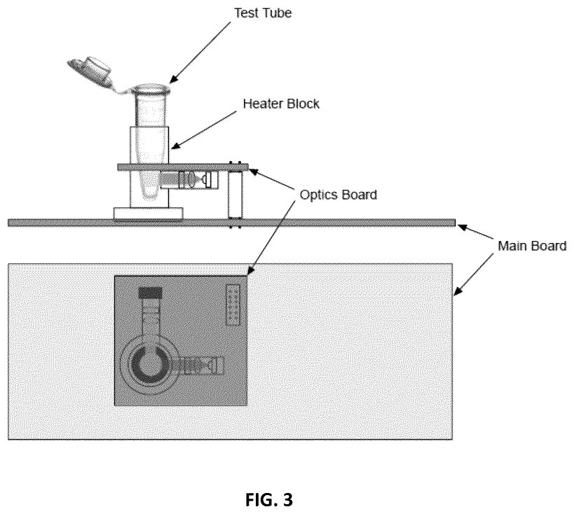

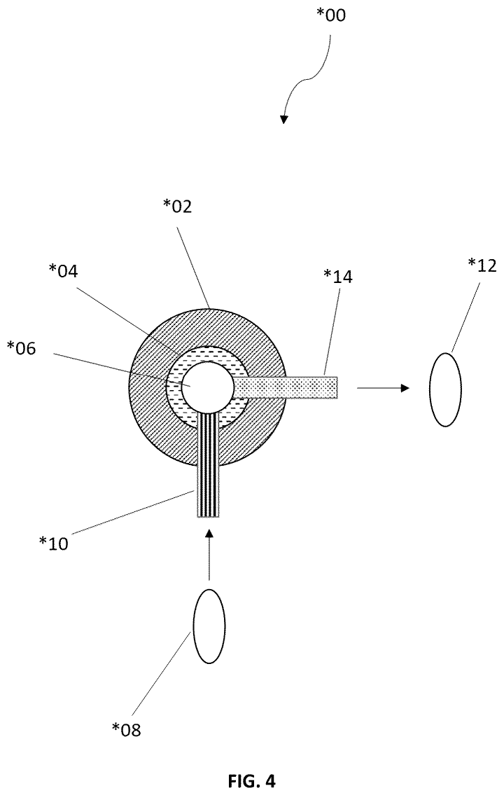

FIG. 2 shows the internal components of the device and FIGS. 3 and 4 provide schematic details. FIG. 4 shows the heating block and reader (00) comprises several elements that are individually numbered. Exemplary elements of the design include a material with a large thermal mass (relative to the mass of the test tube and the materials contained in the test tube may be formed into a block (02). Part of the block (02) may comprise a heating element (04). The heating element 04 may comprise, for example, a resistive heating element or an inductive heating element. The heating element (04) may comprise a thermoelectric heating element. In an embodiment, the heating element (04) may surround or partially surround the test tube (06). In an embodiment, the block (02) may surround the test tube to provide a large mass of material heated to the proper temperature to maintain the temperature desired for the reactions to take place rapidly without degradation of results. In an embodiment, the block or the heater or both may have openings to permit the entrance of excitation light and the exit of fluorescent light. In the example in FIG. 4, the optical excitation source (08) may comprise a laser or a light emitting diode or some other light source. The excitation light may pass through an optical element (10) before entering the test tube (06), where it may excite the dye, causing the dye to emit light, under the appropriate conditions. The emitted light may pass through an optical element (14) before entering a detector or sensor (12).

In an example, the sensors may measure the intensity of the emitted light after a set period of time. In an example, the sensors may begin to measure the intensity of the emitted light after the sample has reached a certain temperature, for instance, 65° C. In an example, the sensors may measure the intensity of the emitted light every 20 seconds. In an example, the period between measurements may vary depending on various factors: total time elapsed since mixing the reagents, time since the heating element reached a temperature threshold, after a number of heating and cooling cycles, or other factors. In an example, a baseline intensity may be measured prior to heating the sample.

### V. Magnetic Bead and Stick Method

Disclosed herein is a simplified method comprising silica-coated magnetic beads and magnetic sticks for rapid extraction of DNA or RNA from various pathogens and sample matrices for direct use in target gene amplification reactions. The overall sample preparation process involves four main steps: (1) lysis (release of nucleic acids from microbe), (2) binding the nucleic acid to the silica on beads, (3) washing the beads to remove contaminants, and (4) elution of the nucleic acids into a buffer compatible with target gene amplification.

In one embodiment lysis and nucleic acid binding are combined in a single step. In this lysis/binding step, a lysis buffer is combined with silica-coated magnetic beads and the sample matrix (e.g., serum, feces, enriched culture).

Also disclosed is a magnetic stick comprising a low-density polyethylene plastic body having a small neodymium magnet adhered to the distal end. In one embodiment, the magnetic stick is submerged in the lysis buffer and after a short 5-minute incubation at room temperature, the magnetic beads (containing microbial nucleic acids) are bound to the magnet surface of the stick.

In some embodiments, the body of the magnetic stick is composed of low-density polyethylene, high density polyethylene, polypropylene, polyethylene terephthalate, polystyrene, polyvinyl chloride, polyoxymethylene, acrylic (polymethyl methacrylate), polyurethane, polycarbonate, polytetrafluoroethylene, polyetherimide, polyether ether ketone, nylon (polyamide), bamboo, wood, or metal (iron, aluminum, zinc, copper, magnesium, and alloys thereof).

In one embodiment, the body of the magnetic stick is between 100 mm to 200 mm in height. In another embodiment the body of the magnetic stick is between 1 mm to 5 mm in diameter.

In other embodiments, the magnet is composed of neodymium, iron-nitride, tetraenite, hematite, magnetite, maghemite, alnico, ferrite, samarium cobalt, magnetic rubber, ilmenite, ulvospinel or electromagnet.

In one embodiment, the magnet between 1 mm to 5 mm in diameter. In another embodiment the magnet is between 1 mm to 5 mm in height. In other embodiments the magnet may be in different shapes, including but not limited to cylindrical, square, or rectangular shapes.

Another embodiment presents a simplified method of washing of the magnetic beads. Commercial test kits typically use 2 or more washing steps to remove contaminants from the magnetic beads that can interfere with the target gene amplification enzymes. In one embodiment, the magnetic stick carrying the silica coated beads is dipped into a wash tube for only 10 seconds to remove any residual components of the lysis buffer. As a final step, the magnetic stick is transferred to an elution tube for 5 minutes, where the nucleic acids are released from the silica beads. Once elution is complete, the magnetic stick and associated beads are discarded. The elution tube now contains concentrated pathogenic nucleic acids, which can be transferred to a tube containing dried target gene amplification reagents for DNA/RNA amplification.

Compared to a commercial DNA/RNA extraction kits, the magnetic bead and stick method disclosed herein has many benefits: (1) rapid lysis of various pathogens and matrices at room temperature in less than 20 minutes without the need for additional equipment, (2) separation of nucleic acids from cellular and sample components, (3) simple composition of lysis, wash, and elution buffers, (4) direct mixing of the elution buffer with dried target gene amplification reagents, (5) overall processing time of less than 30 minutes, and (6) no additional equipment is required, so the process can be done at Point-of-Care (POC). Unlike the existing commercial process, the disclosed magnetic bead and stick method uses wash and elution steps where the beads remain bound to the magnet. It is advantageous to leave the beads bound to the magnet to enable rapid sample processing and decrease the chance of error by the end user.

The use of the disclosed magnetic bead and stick method can extend to many applications such as the detection of viruses in serum, detection of parasites washed off of fruits and vegetables, and the detection of bacteria in fecal samples. In one embodiment, the method can be especially useful for the detection of microbes in environmental samples where the target organism is present in low concentrations. Environmental samples are typically composed of large volumes that are too dilute to be amplified directly. The magnetic stick and bead method inherently concentrates nucleic acids during the process, which contributes a significant increase to sensitivity. Increasing sensitivity is particularly important for environmental samples where less than 10 bacteria may reside in a large sample volume.

In another embodiment, the magnet on the magnetic stick attracts magnetic beads designed to bind nucleic acids released from biological samples. In some embodiments, the sample is selected from a group consisting of serum, plasma, feces, urine, blood, oral fluids, bile, milk, colostrum, nasal secretions, oral secretions, ocular secretions, and fluids from tissues derived human, animal, or other multicellular, complex species at risk for microbial diseases. In another embodiment, the sample is minced, ground, mashed or similarly processed meat, fruits, vegetables, fish, bottled beverages, or other processed food products with a risk of contamination with microbes. In another embodiment the sample is collected from an environmental surface at risk from contamination by infected animals or foods; or a sample of water used to wash animals, foods, or environmental surfaces. In some cases, the sample is taken from an enriched culture, wherein the enriched culture is derived from the aforementioned samples.

Another aspect presents a method of diagnosing an infection within a subject, wherein the magnetic stick is used to attract magnetic beads containing nucleic acids or microbes from the biological sample of the subject.

Yet another aspect presents a method of detecting contamination in a food source, wherein the magnetic stick is used to attract magnetic beads containing nucleic acids or microbes derived from the biological sample collected from a food source.

In another aspect, the invention relates to a method of processing a sample for direct use in target gene amplification reactions comprising: a) lysing the sample to release of nucleic acids from microbe; b) binding the nucleic acids to silica-coated beads; c) incubating the nucleic acid bound silica-coated magnetic beads with a magnetic stick to allow the binding of the silica-coated magnetic beads to the magnetic stick; d) washing the beads on the magnetic stick to remove contaminants; e) eluting the nucleic acids bound to the magnetic stick into a buffer compatible with target gene amplification; f) transferring the eluted nucleic acids to a tube containing dried target gene amplification reagents for DNA/RNA amplification.

In one embodiment, lysis of the sample and binding of the released nucleic acids to silica-coated beads are completed in a single step. The sample is incubated with a lysis buffer combined with silica-coated beads and the sample matrix for 5 minutes at room temperature. In some embodiments, the lysis buffer contains chaotropic agents (urea, guanidinium salts), organic solvents (methanol, ethanol, propanol, butanol, acetone), enzymes (lysozyme, proteinase K), amino acids or polypeptides, buffering agents (Tris, HEPES, carbonate, phosphate, glycine, tricine, bicine, PIPES), or detergents (SDS, SLES, CHAPS, CHAPSO, n-octyl-beta-D-glucopyranoside, Triton X-100, Triton X-114, polysorbate (Tween 20 or Tween 80), CTAB). In other embodiment, the magnetic stick bound with the silica coated beads is dipped into a wash tube for 10 seconds to remove residual components of the lysis buffer and sample matrix. The magnetic stick is then transferred to an elution solution for 5 minutes, wherein the nucleic acids are released from the silica-coated beads. In some embodiments, the sample includes biological material selected from a group consisting of serum, feces, urine, blood, oral fluids, processing fluids, environmental samples, nasal secretions, oral secretions, ground meat, produce, fish, liquid drinks and foods, and enriched culture.

In yet another aspect, the invention relates to a rapid method for the direct amplification of target genes from milk comprising; a) lysing a milk sample by heat; b) adding the lysed sample to target gene amplification reagents in a PCR tube; c) adding a chemical to prevent non-specific amplification (e.g., polyaspartic acid, polyglutamic acid) to the sample in the PCR tube; and e) running a target gene amplification reaction. In some embodiments, the milk is colostrum, whole milk, reduced fat milk, skim milk, buttermilk, powdered milk, condensed milk, or evaporated milk.

In another aspect, the invention relates to a sample enrichment method comprising: a) mixing a sample with an ion exchange resin that can bind the microbe of interest; b) separating the ion exchange resin by centrifugation, filtration, or magnetic force; c) incubating the resin-bound sample in a lysis buffer to concentrate and extract nucleic acids into a smaller volume; d) adding the extracted DNA or RNA to target gene amplification reagents in a PCR tube; and e) running a target gene amplification reaction.

In some embodiments, the exchange resin is a positively charged anion exchange resin wherein the anion exchange resin is incubated with a negatively charged sample. In some other embodiments, the exchange resin is a negatively charged cation exchange resin wherein the cation exchange resin is incubated with a positively charged sample. In other embodiment, the sample is selected from bacteria, parasites, viruses, fungi, allergen, DNA, or RNA. In other embodiments, the anion exchange resin functionality is a weakly basic anion (primary, secondary, and tertiary amines like dimethylaminopropyl, polyethyleneimine, or diethylaminoethyl) or a strongly basic anion (quaternary amines like diethyldialkyl ammonium chloride or alkyldialkyl ammonium chloride). In other embodiments, the cation exchange resin functionality is a weakly acidic cation (carboxylic acids) or a strongly acidic cation (sulfonic groups).

In another embodiment, the magnetic beads separated from other components in the sample by magnetic force.

In yet another aspect, the invention relates to a method of concentrating bacteria from dilute samples comprising: a) loading a sample into a first syringe, b) attaching a 0.45, 0.22, or 0.1 micron syringe filter; c) depressing the plunger on the first syringe to force the liquid through and thereby trapping the sample on the syringe filter; d) transferring the syringe filter with the trapped sample to a second syringe containing a smaller volume of lysis buffer; e) passing the lysis buffer through the second syringe and the first syringe filter; thereby releasing sample genomic nucleic acids; and f) adding the eluted genomic nucleic acids to a target gene amplification reaction for pathogen detection; wherein the method is completed in 5 minutes or less. The syringe filter may be composed of materials such as nylon, polyethersulfone, cellulose acetate, regenerated cellulose, polypropylene, glass fiber, ceramic, metal, or wood.

### VI. Method to Prevent False Positive Amplification

Milk and its by-products is an economically important commodity accounting for over $36 billion in annual US sales. However, milk can harbor many dangerous pathogens, such as Campylobacter, Cryptosporidium, E. coli, Listeria, Brucella, and Salmonella. Rapid point-of-care diagnostics are a critical tool for milk manufacturers to ensure the safety of their product from these and other pathogens. Milk is also tested by veterinarians to diagnoses mammary gland infections (mastitis) that decrease milk production, make milk unsuitable for use due to high somatic cell counts, and can be deadly for the cow. Rapid diagnosis of the microbe causing mastitis (bacteria, yeast, or algae), is important to selecting the proper drug to treat the infection. One challenge to implementing rapid POC diagnostics for the detection of milk pathogens is the composition of milk itself—a high protein and high fat matrix that can complicate sample preparation procedures.

A challenge is using milk-containing products in target gene amplification reactions is that once the sample has been added to a target gene amplification reaction, the protein and fat components lead to irregular and unpredictable amplification in the absence of target (i.e., false positive amplification). Disclosed herein is a method that enables direct addition of milk samples to target gene amplification reactions. In one embodiment, a polyamino acid (polyaspartic acid (PLD), MW 1,400) is added to the target gene amplification reaction to control false positive amplification in the presence of milk components. This polymer reduces false positive amplification by binding to the polymerase and preventing non-specific interaction with the primers. Polyglutamic acid (PLE) is a second chemical that may also diminish non template amplification. PLD and PLE can be useful for other challenging samples, not just milk.

### VII. Direct Capture of Bacteria by Anion Exchange Resin

Environmental samples are a routinely collected in industrial food processing settings. These sample types are usually quite dilute, sometimes containing less than 10 bacteria in multiple milliliters of solution. Detection of specific pathogens in these extremely dilute samples can be quite challenging, and current methods employ lengthy overnight bacterial culture methods to enrich bacterial populations. For some microbial targets, enrichment in synthetic growth media is not always possible, further limiting the application of diagnostic processes relying on bacterial enrichment methods.

Disclosed herein is an enrichment method based on anion exchange chromatography that can be performed in less than 15 minutes to overcome the limitations of current bacterial enrichment culture methods. In one embodiment, the method comprises a strong positively charged anion exchange functionality (quaternary amine) that binds to negatively charged molecules on the outer membrane of bacteria. Once bound to bacteria, the anion exchange resin can be easily separated by centrifugation, filtration, or magnetic force if using magnetic beads. Incubation of the resin and bound bacteria in a lysis buffer enables both concentration and extraction of DNA into a smaller volume that is easily handled in diagnostic processes. Careful selection of the lysis buffer formulation allows direct addition of the eluate to a target gene amplification reaction for detection of pathogens. The disclosed method has many advantages over current concentration and enrichment methods, like bacterial culture, such as: (1) completion of the method in less than 15 minutes compared to bacterial culture that often requires 18+ hours, (2) separation of the bound bacteria from unwanted sample matrix components, and (3) potential to capture any target with negatively charged functionalities, like bacteria, parasites, viruses, fungi, allergens, DNA, or RNA, which cannot always be easily cultured.

### VIII. Concentration of Bacteria from Dilute Samples Using a Syringe Filter

The food industry relies on extremely sensitive pathogen detection technologies to ensure that food products are safe for consumers. Sensitive detection of pathogens is especially important for environmental samples taken from the surfaces surrounding food production areas, since these samples may contain less than 10 bacteria in a 10 cm×10 cm sampling area. Due to the rapid multiplication of bacteria every 20 minutes under the right conditions, even a small number of bacteria can quickly surpass a minimal infectious dose. Current diagnostic methods use this rapid growth to enrich bacterial populations in food samples in a method known as “bacterial culture” (or just simply “culture”). Increasing the total number of bacteria in a sample greatly simplifies downstream detection assays. The biggest issue to using culture methods is the time involved. Typically, these cultures are grown over 18 hours to maximize the number of bacteria from a sample. Release of food products or manufacturing facilities may depend on results from these time-consuming diagnostic processes. Long sample processing times, like overnight culture methods, reduce the shelf-life of food products, which may be as low as 3 to 5 days for some fresh meat products (A. K. Magoulas & CiCi Williamson, USDA's Food Safety and Inspection Service in Health and Safety, Aug. 19, 2014). Recertification of contaminated work areas in processing plants may require the successful completion of at least three consecutive negative tests. When culture is required, these replicate tests may take 7 to 10 days, which is lost time and impacts plant capacity/profitability. Innovations that shorten test time without compromising sensitivity would be a welcome improvement.

Disclosed herein is a novel bacterial concentration method that can be performed in less than 5 minutes, thereby avoiding long sample processing times for bacterial targets. The method uses a syringe filter to capture and concentrate bacteria from dilute samples. In one embodiment, the method comprises loading an environmental sample into a syringe and then connecting a 0.22-micron syringe filter (alternatively 0.45 or 0.1 micron). Depressing the syringe plunger forces the liquid through the filter. However, the bacteria are too large to pass through the filter and instead remain on the filter surface. After the entire sample has passed through the filter, the syringe filter containing the trapped bacteria is transferred to a new syringe containing a smaller volume of lysis buffer. The lysis buffer is formulated to ensure rapid breakdown of bacteria. When the plunger on the second syringe is depressed, the genomic DNA from the lysed bacterial cells passes through the filter with the buffer. The buffer and eluted DNA from the filter can be directly added to a target gene amplification reaction for pathogen detection. The disclosed method poses many advantages compared to current bacterial culture methods, such as: (1) completion of the method in less than 5 minutes compared to the 18+ hours needed for bacterial culture, (2) separation of the target bacteria and nucleic acids from unwanted sample matrix components, (3) lysis and concentration of bacteria into a volume easily handled in target gene amplification reactions, and (4) potential to capture any target larger than the syringe filter pore size, like bacteria, parasites, viruses, fungi, and allergens, which cannot always be easily cultured.

### EXAMPLES

### Example 1: Dried Target Gene Amplification Reagents—Vacuum Method

### Materials and Methods

Unless otherwise specified, the following Materials and Methods were used in the Examples below: Enzyme and buffer components used in the reactions were from the 2× LAMP Master Mix from New England Biolabs (NEB). The term “target gene amplification reagents” typically refers to this pre-made solution containing components such as the WarmStart Bst 2.0 polymerase, WarmStart RTx reverse transcriptase (RT), buffering components, dNTPs, magnesium sulfate, Tween-20, and other necessary components included by NEB. The test PRRS virus was reconstituted Ingelvac PRRS MLV from Boehringer Ingelheim. A positive control DNA gBlock was synthesized by IDT and matched the sequence of the primer set. An intercalating fluorescent dye was used at 1× in the reverse transcriptase target gene amplification and target gene amplification reactions and is either the 50× target gene amplification fluorophore (NEB) or 20× EvaGreen Plus (Biotium). Serum was separated by centrifugation from blood collected from swine. Unless otherwise specified, all reactions below were incubated at 65° C. in a CFX96 qPCR instrument (Bio-Rad) and measured for fluorescence using the blue excitation and green emission wavelengths (“FAM” or “SYBR Green” channel).

To preserve the stability of target gene amplification reagents at room temperature (18° C. to 30° C.) and simplify the format of a commercial diagnostic kit, the target gene amplification reagents can be dried in the final reaction tubes. A protective drying agent, such as trehalose, sucrose, or dextran can be added to protect the structure of a protein during the drying process. Two methods to dry proteins include the use of vacuum (low pressure) at room temperature and dry heat (40° C. to 60° C. without vacuum). target gene amplification reagents and target gene amplification primers were physically separated to two separate locations on the PCR tube during the drying process to minimize false positive amplification, which can be enhanced by the drying process.

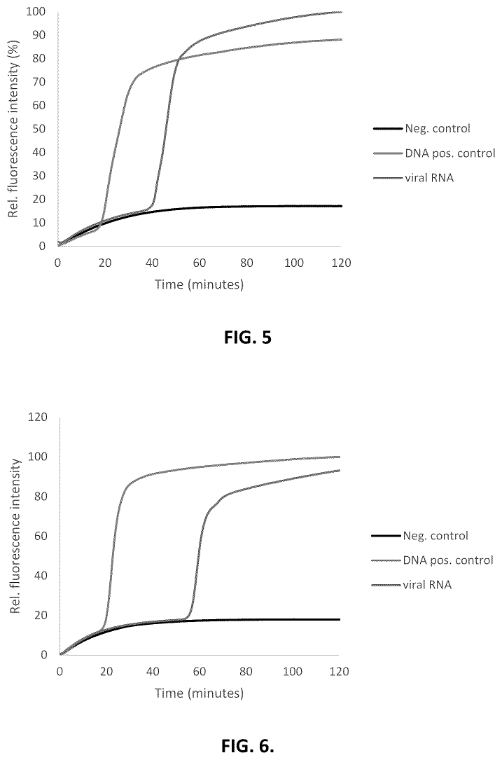

To test the vacuum drying process, target gene amplification reagents were adjusted to 5% sucrose, 8% trehalose, 0.9% dextran, or 0.9% dextran plus 2.5% glycerol and added to a PCR tube. Target gene amplification primers were also adjusted with the same stabilizers and added to the same PCR tube but in a physically separate location. After drying for 23 hours at room temperature under vacuum, a liquid sample containing a DNA positive control (synthetic DNA containing gene sequence to be amplified), water (negative template control), or in some cases an uncoated PRRS virus in vaccine diluent was used to resuspend the dried target gene amplification reagents and primers. The primers used are listed in Table 1. Target gene amplification reactions were conducted at 65° C. and detected by intercalation of a fluorescent green dye into the DNA products (FIGS. 5 to 8). PGP-21 DNA

TABLE 1
Sequences of primer sets for the detection of the
PRRSV ORF7 genetic target.

| | SEQ ID |
|---|---|
| Sequences | NO |
| TGCCGTTTGTGCTTGCTAG | 1 |
| AGGTTTACCACTCCCTGCT | 2 |
| ATCGGATGAAAGCCTGCGGCGCCGCAAGTACATTCTGGC | 3 |
| TAACCACGCATTTGTCGTCCGGCAACACGAGGCTTTTCAACC | 4 |
| ACTTTCAACGTGGTGGGC | 5 |
| GGCTCCACTACGGTCAACG | 6 |

An alternative primer set for the detection of the PRRSV ORF7 genetic target is provided in Table 2. The ORF7 consensus sequence was compared to 1,000 separate PRRSV strains using the Basic Local Alignment Search Tool (BLAST). Changes from the original sequences that target gene amplification primer sequences are shown in bold, underlined, and enlarged letters.

TABLE 2
Sequences of alternative primer sets for the
detection of PRRSV.

| | SEQ |
|---|---|
| Sequences | ID NO |
| TGCCGGTTGTGCTTGCTAG | 7 |
| AGGTTCATCACTCCCTGCT | 8 |
| ATCGGATGAAAGCCCGCGGCGCCGCGAGTACGTTCTGGC | 9 |
| TAACCACGCATTTGTCGTCCGGCAACACGAGGCTTTTTAACC | 10 |
| ACTTTCGACGTGGTGGGC | 11 |
| GGCTCTACTACAGTTAACG | 12 |

### Results

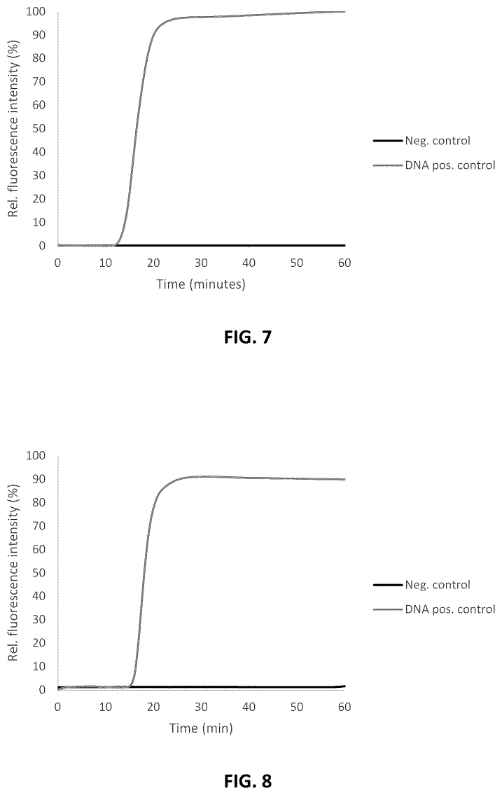

The results showed that PRRSV RNA and the positive control could be amplified using vacuum-dried target gene amplification reagents stabilized with 5% sucrose (FIG. 5) and 8% threhalose (FIG. 6). The negative control showed only background fluorescence at <20% of the maximum signal, confirming that primers dried separately from the enzymes did not result in false positive amplification. These results confirm that drying under vacuum using these two stabilizers did not destroy the enzymatic activity of the Bst enzyme or the reverse transcriptase enzyme. Vacuum drying of the target gene amplification reagents also showed excellent amplification of the DNA positive control when stabilized with 0.9% dextran (FIG. 7) or 0.9% dextran plus 2.5% glycerol (FIG. 8). target gene amplification target gene amplification target gene amplification reagents and primers dried under vacuum using these stabilizers also showed no fluorescent signal, which is attributed to drying of the DNA primers in a separate location to prevent primer bonding with enzymes during drying that could result in false positive amplifications. Additional drying agents may be investigated in the future to improve the speed or fluorescence intensity of the target gene amplification reaction.

### Example 2: Dried Target Gene Amplification Reagents—Dry-Heat Method

### Materials and Methods

To test the dry-heat process, target gene amplification reagents were adjusted to 5% sucrose or 8% trehalose and added to a PCR tube. target gene amplification primers were also adjusted to the same concentration of drying agent and added to the same PCR tube but in a physically separate location. The tubes were placed in a dry-heat oven at 45° C. for 1.5 hours. After drying, a liquid sample containing uncoated PRRS virus, the DNA positive control in water, or water (negative control) was used to resuspend the dried target gene amplification reagents. target gene amplification reactions were conducted at 65° C. and detected by intercalation of a fluorescent green dye into the DNA products.

### Results

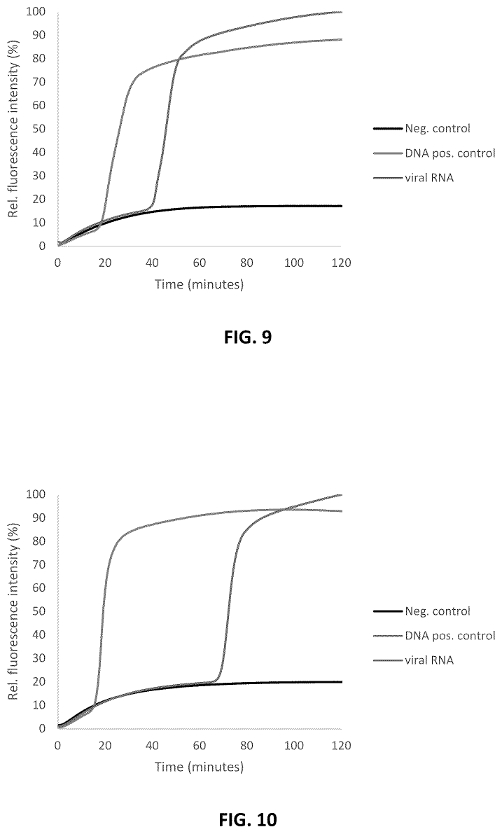

The results showed that both DNA positive control and viral RNA could be amplified using target gene amplification reagents heat-dried in 5% sucrose (FIG. 9) or 8% trehalose (FIG. 10). As found before, the drying of primers separately from the target gene amplification reagents under these conditions also resulted in no false positive amplification. Simple variations in time and temperature for drying may result in improved target gene amplification speeds.

### Example 3: Viral Lysis at Low Temperature

Effective viral lysis that sufficiently exposes viral RNA to a reverse transcriptase is necessary to the detection of viruses by RT-modified target gene amplification. Heat is commonly used to denature capsid proteins and expose viral RNA. However, the use of very high temperatures (e.g., 95° C. for 1 minute) can have detrimental effects for the stability of both the viral RNA and sample matrix. For serum samples the use of high temperatures can cause serum proteins to denature and aggregate, which makes the transfer of the sample to an RT-modified target gene amplification reaction difficult due to an extremely viscous and gelatin-like state. Experiments were conducted to determine whether viral uncoating would occur at temperatures between 95° C. and 65° C. (the temperature used for target gene amplification reactions). These experiments indicated feasibility for incubation at 75° C., which was confirmed in the following experiment.

### Materials and Methods

To lyse virus in serum at low temperature, a sample of serum containing PRRS virus (as described in Example 1) was diluted to a final concentration no greater than 50% in water and heated in a tube at 75° C. for 5 minutes. After heating for 5 minutes, the serum sample was allowed to cool to room temperature then added to a pre-assembled RT-modified target gene amplification reaction so that the final concentration of serum was 25%. After thoroughly mixing, the RT-modified target gene amplification reaction was incubated at 65° C. and measured for fluorescence.

### Results

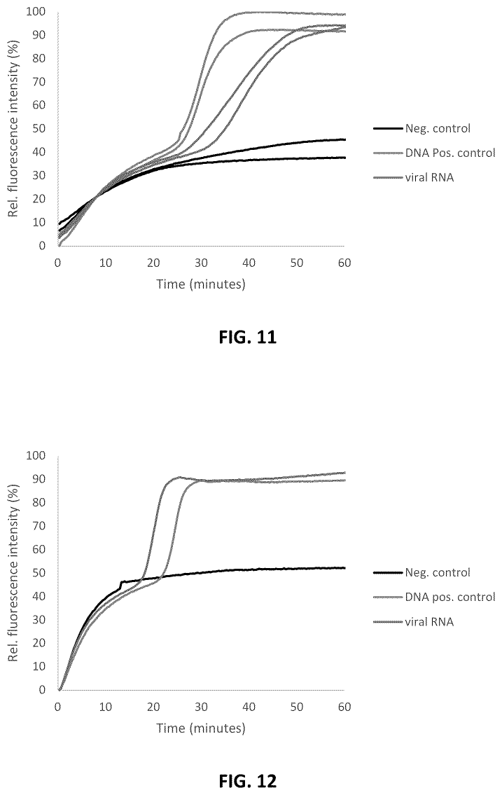

Heating serum to 75° C. for 5 minutes did not result in significant coagulation/gelling of serum proteins and resulted in successful amplification of viral RNA within 40 minutes (FIG. 11). The amplification was slower than the DNA positive control but achieved the same level of intensity. No false positive reaction was detected in the negative control. A temperature of 75° C. is very close to the maximum reaction temperature of 70° C. reported for some Bst and RT enzymes. Therefore, careful selection of target gene amplification enzymes and primers may enable viral lysis plus amplification to occur simultaneously in a single tube. The simplicity of diluting serum into a target gene amplification reaction tube, placing the tube into a device, and waiting for results is ideal for a POC test conducted at a farm.

### Example 4: pH-Based Viral Lysis with Aqueous Target Gene Amplification Reagents

The use of lower heat prevented gelling of serum proteins and facilitated proper uncoating of virus in diluted serum. However, this method may require incubation of the diluted sample at 75° C. and then transferring to a separate tube for the target gene amplification reaction at 65° C. A method with a simpler process where viral uncoating could be accomplished at room temperature and without additional equipment was desired. Therefore, chemical treatments to facilitate virus uncoating were investigated. One theory tested was whether serum could be adjusted to a pH above the isoelectric point of the capsid protein. If possible, this pH should disrupt ionic bonding of capsid proteins would enable sufficient uncoating of viruses in serum at room temperature.

### Materials and Methods

To perform the pH-based viral lysis, a sample of serum containing PRRS virus (per Example 1) was diluted to 25% in lysis buffer composed of 14 mM sodium hydroxide (1 volume of serum plus 3 volumes of lysis buffer). After a 5-minute incubation at room temperature, a portion of the diluted serum in lysis buffer was added to a tube containing pre-mixed liquid RT-modified target gene amplification reagents, so that the final concentration of serum was 6.25% and mixed well. The tube was then incubated at 65° C. to initiate the RT-target gene amplification reaction and fluorescence generated by the amplification was measured. In separate experiments, Tween-20 (0.1%) or Tween-20 (0.1%) and Triton X-100 (0.1%) were added to the lysis buffer to determine whether these detergents would enhance viral uncoating. The target gene amplification reagents, positive control, negative control used, and reaction conditions were the same as described in Example 1.

### Results

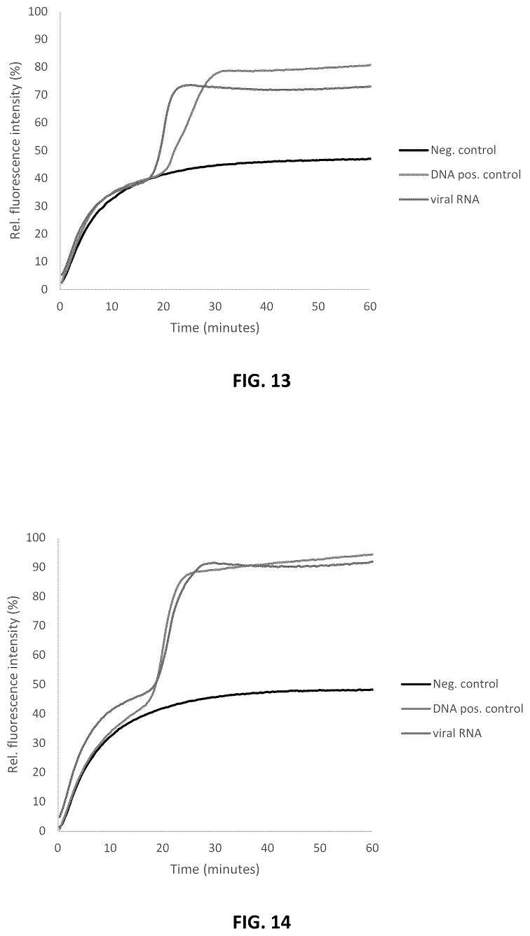

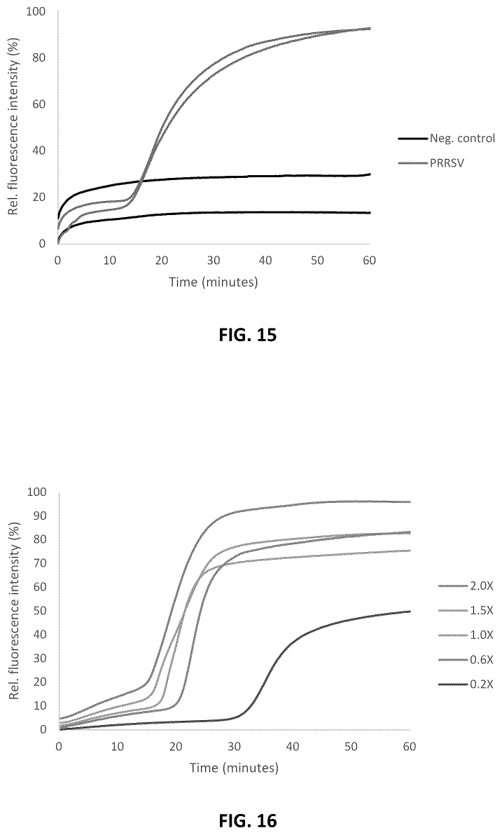

Raising the pH of serum to ˜11 successfully uncoated the PRRS virus as shown by amplification in ˜20 minutes with an intensity matching the DNA positive control (FIG. 12). The amplification of viral RNA was rapid, occurring within a similar time as a DNA positive control. The addition of detergents to the lysis buffer, like Tween-20 (FIG. 13) or a combination of Tween-20 and Triton X-100 (FIG. 14), did not significantly change the time to a positive reaction. These data show that this level of detergent(s) does not interfere with Bst and RT enzymatic reactions, therefore, this method may be useful for uncoating of different viruses with varying envelope compositions.

### Example 5: pH Optimization of Serum Samples/Target Gene Amplification Reactions

The polymerase typically used in target gene amplification reactions is Bst, which has an optimal activity (highest enzymatic activity) at pH 8.8. To maintain a pH of 8.8, target gene amplification reagents are typically buffered with Tris-HCl. If serum adjusted to high pH for viral lysis is directly added to dried target gene amplification reagents, the high pH could overwhelm the standard Tris buffer, resulting in slow amplification or as a worst case causing the Bst enzyme to be denatured and non-functional. Therefore, the addition of Tris-HCl with a higher molarity was tested to determine whether it would result in improved amplification of viral samples lysed using high pH in serum.

### Materials and Methods

A swine serum sample was inoculated with PRRSV vaccine virus to final concentration of 10⁶ particles/mL. The PRRSV serum sample (30 uL) was added to 60 uL 14 mM NaOH, mixed, and incubated at room temperature for 5 minutes. After the incubation, 30 uL of a neutralization buffer containing 80 mM Tris, pH 8.8, fluorophore, and primers was added to the sample and mixed. From this neutralized mixture, 30 uL was added to dried RT-modified target gene amplification reagents in a PCR tube. After resuspension, the target gene amplification reactions were incubated at 65° C. and fluorescence generated by the amplification was measured.

### Results

Error! Reference source not found. 15 shows a fluorescent intensity graph of the amplification of viral RNA obtained by the pH lysis method. As expected, no amplification occurred in the absence of nucleic acid sequences (negative control, black lines). Target gene amplification reactions assembled with viral RNA prepared by the pH lysis method enabled rapid detection of PRRSV.

### Example 6: Optimization of Primer Concentrations

The target gene amplification of DNA relies on 6 separate primers. The outside primers (forward and reverse) flank the entire gene segment that is targeted for amplification. The outside primers are used by the reverse transcriptase to convert an RNA-based gene of interest from RNA into a cDNA copy for target gene amplification. The four internal primers create a dumbbell-like structure that can continuously be amplified by the target gene amplification strand-displacing polymerase. Since each primer set and target gene are unique, the concentration of primers for an optimal target gene amplification reaction should be optimized for each target.

### Materials and Methods

Multiple different mixtures of primers were tested (Table 3). Each mixture was diluted in a target gene amplification reaction containing the necessary components for amplification. One set of experiments used uncoated PRRS virus added directly to the target gene amplification reaction containing primer mixes varying from 2× to 0.2×. A second experiment tested the DNA positive control spiked into serum and amplified with primer mixes varying from 2× to 0.2×. Apart from the varying primer concentrations, the target gene amplification reactions were conducted and detected as described in Example 1.

TABLE 3
1X Mixture of Primers

| Primers | Mix 1 | Mix 2 | Mix 3 |
|---|---|---|---|
| FIP & BIP | 1.6 μM | 1.6 μM | 1.6 μM |
| FL & BL | 0.4 μM | 0.8 μM | 0.8 μM |
| F3 & B3 | 0.2 μM | 0.2 μM | 0.3 μM |

### Results

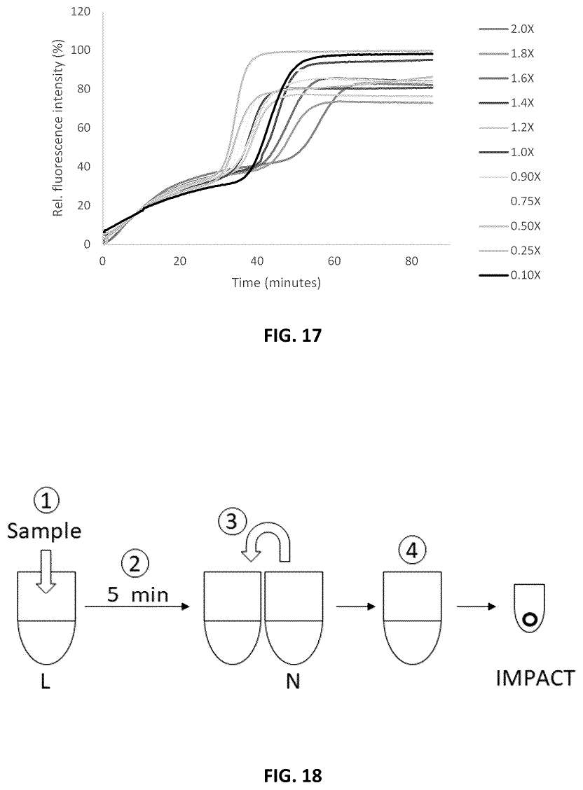

An example titration of primers for the amplification of viral RNA is shown in (FIG. 16). The results indicated that the speed and intensity of amplification was not significantly different until the primer set was diluted to 0.2×. In the case of amplification in the presence of serum, FIG. 17 shows quite different and unexpected results from the primer titration. In this case, lower levels of primers resulted in faster amplification and the 2× primers were the slowest. These data indicate the importance of primer optimization to improve the reaction speed and fluorescent intensity when testing in the presence of different complex biological samples. Also, the finding that more primers is not necessarily better can be used to reduce costs.

### Example 7: PRRSV/Serum Workflow (with Neutralization Buffer)

A commercial workflow is outlined in FIG. 18 for the preparation of viral nucleic acids from various sample types. Step 1 involves addition of a collected sample to a lysis tube containing high pH lysis buffer. The sample is incubated in lysis buffer for 5 minutes at room temperature (Step 2). A drop of neutralization buffer (N) is added to the lysis tube (L) using a commercially available pipettor and mixed by inversion (Step 3). After neutralization, nucleic acid in the lysis tube is transferred to the amplification reaction containing dried target gene amplification reagents (buffers, nucleotides, fluorescent dye and Bst enzyme for all targets; and additional RT enzyme for RNA viral targets) and primers (Step 4).

The reaction tube is then inserted into an instrument that can maintain 65° C. and measure fluorescence every 20 seconds for 2 hours (excitation 470-490 nm, emission 510-530 nm). For example, a Bio-Rad CFX96 qPCR instrument or a prototype target gene amplification device.

### Example 8: Target Gene Amplification of Five Separate Salmonella Serovars

Salmonellosis is one of the most common foodborne illnesses and is caused by infection with the bacterium Salmonella enterica. However, there are over 2,600 serotypes of Salmonella. Detection of these different serotypes by target gene amplification is challenging due the variety of genetic differences between these serotypes. To create a primer set specific for Salmonella and also able to amplify diverse serovars, a highly conserved genetic target must be used. For Salmonella, one such highly conserved gene is invA, which encodes for the invasion gene necessary for Salmonella virulence. A novel primer set was developed following genetic analysis of 100 genomes from different Salmonella strains to identify a highly conserved region for amplification. A consensus sequence for the invA gene was created from this data set and used to create multiple primer sets of 5-6 primers each. The primer sets were initially screened with a DNA positive control to ensure each primer set was functional and did not amplify in the absence of nucleic acid template (data not shown). After the initial screen, a top-performing primer set was chosen for further evaluation.

### Materials and Methods

Genomic DNA (gDNA) from five different serotypes of Salmonella enterica subsp. enterica (including serovars agona, Berta, Braenderup, 4,[5],12:i:-, and Typhimurium) were purified from cultures using a commercial DNA purification kit. In the first phase, the purified gDNA from all five serotypes were combined in equal parts and added to target gene amplification reagents. In a confirmatory experiment, the purified gDNA from each serovar was used separately. The four different primer sets targeting the invA gene (Table 4) were tested in separate reactions. The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument.

To confirm the ability of the primer set to amplify individual Salmonella strains/serovars, a target gene amplification reaction was assembled using the invA primer set 2 (Table 4). A separate reaction tube was tested using gDNA from each of five swine Salmonella serotypes/strain: Typhimurium, Berta, 4,[5],12:i:-, agona, or Braenderup (genomic DNA purified by commercial QIAGEN DNeasy Blood & Tissue Kit). Target gene amplification reactions were conducted at 65° C. and detected by intercalation of a fluorescent green dye into the DNA products.

TABLE 4
Sequences of primer sets 1-4 for the detection of Salmonella
invA gene.

| | | SEQ |
|---|---|---|
| Primer | | ID |
| Set | Sequences | NO: |
| 1 | GAACGTGGCGATAATTTCAC | 13 |
| | ATTTCCTGATCGCACTGAA | 14 |
| | CACGCTCTTTCGTCTGGCATTAGCATCGGCTTCAATCAAGA | 15 |
| | ACAGTACCGCAGGAAACGTTGTACTGGCGATATTGGTGTT | 16 |
| | CGATCAGTACCAGTCGTCTTA | 17 |
| | AGGATTCTGTCAATGTAGAACG | 18 |
| 2 | ACACGTTCTGAACCTTTGG | 19 |
| | TACCACGCTCTTTCGTCT | 20 |
| | TTCGGGCAATTCGTTATTGGCCCACGGTGACAATAGAGAAG | 21 |
| | GTGGCGATAATTTCACCGGCGGCATTATCGATCAGTACCA | 22 |
| | GGCGGTGGGTTTTGTTG | 23 |
| | TCGGCTTCAATCAAGATAAGAC | 24 |
| 3 | CGGCAATAGCGTCACCTT | 25 |
| | CGGCCCGATTTTCTCTGG | 26 |
| | GCGAAGCGTACTGGAAAGGGAATAAACTTCATCGCACCGTCA | 27 |
| | GCGCGGCATCCGCATCAATATGCCCGGTAAACAGATGAGT | 28 |
| | GCCAGCTTTACGGTTCCTT | 29 |
| | GCCTTCAAATCGGCATCA | 30 |
| 4 | TCAACAATGCGGGGATCTG | 31 |
| | GAAGCGTACTGGAAAGGGAA | 32 |
| | TGGGGATGACTCGCCATGGTATGACCATCACCAATGGTCAGC | 33 |
| | AATGATGCCGGCAATAGCGTCAGCCAGCTTTACGGTTCCTT | 34 |
| | CCGCCCTGTCTACTTATACC | 35 |
| | ACTTCATCGCACCGTCA | 36 |

### Results

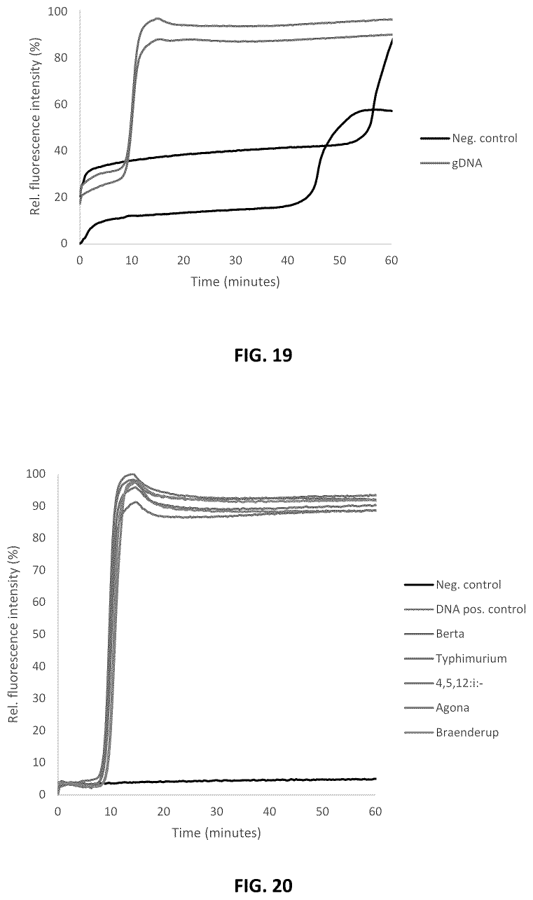

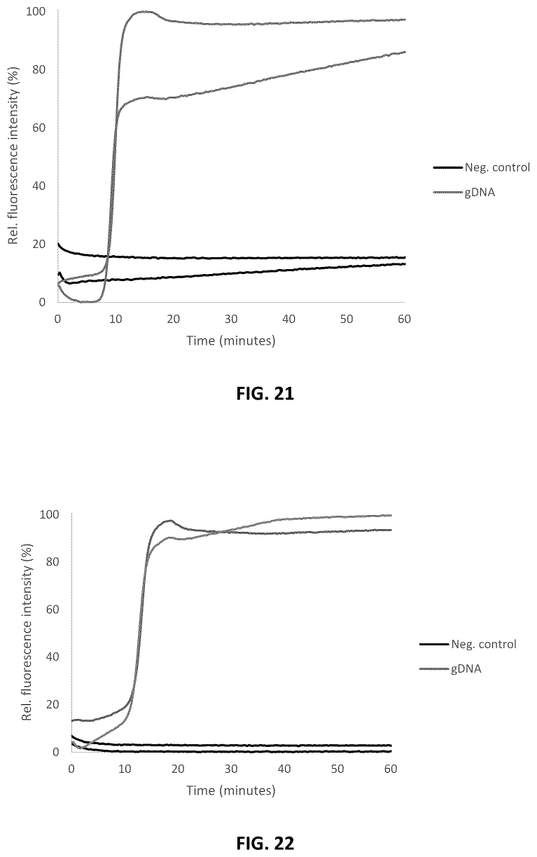

The data confirmed that the mixture of gDNA from the five Salmonella serotypes/strains was amplified in less than 10 minutes with no false positive reactions when using primer set 1 (FIG. 19), primer set 3 (FIG. 21), and primer set 4 (FIG. 22). Similar results were observed with primer set 2 (data not shown). Primer set 2 was selected for further screening using each individual Salmonella strain/serovar in a separate reaction. As shown in FIG. 20, primer set 2 was able to rapidly amplify genomic DNA from five different Salmonella strains in under 15 minutes, and slightly faster than the DNA positive control. Therefore, these primer sets are candidates for commercialization of a test for food safety testing that is expected to detect at least 100 serovars/strains of Salmonella enterica.

Alternative Salmonella primers sets with degenerate base pairs within the original primer set sequences (Error! Reference source not found.5) can also be used. Alternative primer set sequences in this table indicate other probable sequences of invA gene in Salmonella spp. These were found by using BLAST to search for somewhat similar matches to the invA consensus sequence used to originally design the primers. The BLAST returned 1,972 other Salmonella invA sequences for comparison to the consensus sequence. Changes from the original sequences that target gene amplification primer sequences are shown in bold, underlined, and enlarged letters.

TABLE 5
Alternative primer set sequences for detection of the invA gene in
Salmonella spp.

| Primer | | SEQ ID |
|---|---|---|
| Set | Sequences | NO: |
| 1 | GAACGTGGCGATAATTTCAC | 13 |
| | ATTTCCTGATCGCACTGAA | 14 |
| | CACGCTCTTTCGTCTGGCATTAGCATCGGCTTCAATCAAGA | 15 |
| | ACAGTACCGCAGGAAACGTTGTACTGGCGATATTGGTGTT | 16 |
| | CGATCAGTACCAGCCGTCTTA | 37 |
| | AGGATTCTGTCAATGTAGAACG | 18 |
| 2 | ATCCTCCCGAAGCCTTTTG | 38 |
| | TACCACGCTCTTTCGTCT | 20 |
| | TTCGGTCATTTCCTGGTCGGCTAATGACGAGGATGATAAAA | 39 |
| | GTGGCGATAATTTCACCGGCGGCATTATCGATCAGTACCA | 22 |
| | CGCGGTCGGGATTGTTG | 40 |
| | TCGGCTTCAATCAAGATAAGAC | 24 |
| 3 | CGGCAATCGTATCGCCTT | 41 |
| | GCGCCCGATTCGTGCTTG | 42 |
| | GCGAAGCGTCCAGCAAAGGGAATAAATTTCATCGCACCGTCC | 43 |
| | GGGCATCATCTGCACCGATCTGCCCGGTAAACAGATGAGT | 44 |
| | GCCAGCTTTACGGTGCGAT | 45 |
| | GCCTTCAAATCGGCATCA | 30 |
| 4 | TCAACAATGCCGGGATCTG | 46 |
| | GAAGCGTCCAGCAAAGGGAA | 47 |
| | TGGGGATGACCCGCCATGGTATGGCCGTCGCCAATGGTCAGC | 48 |
| | AATAATACCGGCAATAGCGTCAGCCAGCTTTACGGTGCGAT | 49 |
| | CCGCTCTGTCTACTTACACC | 50 |
| | ATTTCATCGCACCGTCC | 51 |

### Example 9: Salmonella in Swine Fecal Sample (Magnetic Bead+Stick Method)

Initial attempts to amplify bacterial DNA from feces spiked with Salmonella were unsuccessful when using dilution and chemical additives to block potential inhibitors. However, the same sample could be amplified if nucleic acids were purified using a complex multi-step process that required vortexing, centrifugation, incubation with proteases, and separation of nucleic acids using a spin column in a centrifuge. This process was incompatible with POC testing due to the scientific skills and equipment required. Therefore, experimentation with various DNA binding matrices led to a simplified method using silica coated magnetic beads.

### Materials and Methods

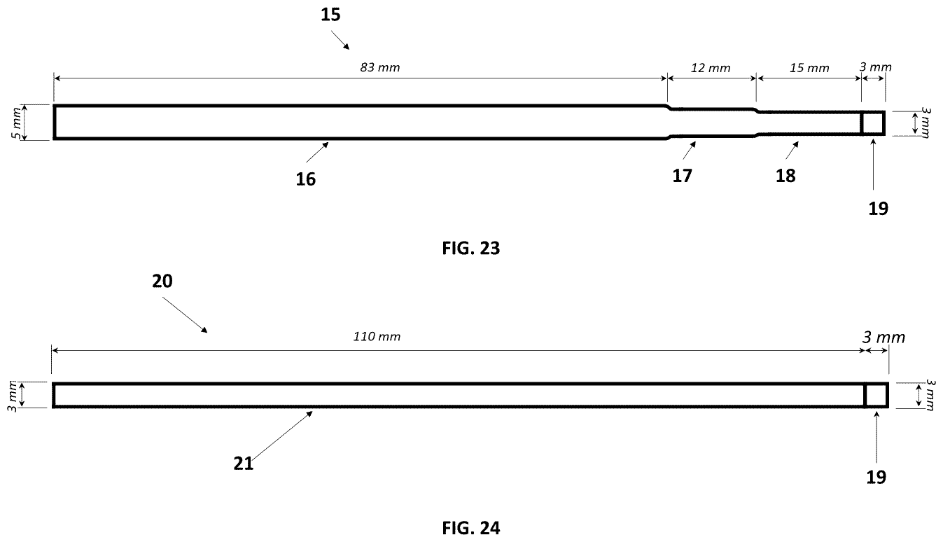

A sample of swine feces (˜200 mg) was obtained that did not contain detectable amounts Salmonella. To this fecal sample was added Salmonella enterica subsp. enterica serovar Typhimurium (10 μL of sample for 100,000 cfu total), and the sample was added to a tube with 570 μL lysis buffer and silica-coated magnetic beads (20 μL of a 50% slurry). The sample was incubated at room temperature for 5 minutes. A magnetic stick (see FIGS. 23 and 24) approx. created by filling the end of a plastic cylinder (approx. 127 height×3 mm diameter) with fast-drying epoxy resin, onto which was placed a small neodymium magnet (3 mm diameter×3 mm height cylinder). The magnetic stick was added to the lysis buffer to bind and collect the magnetic beads that had captured DNA released from lysed bacteria. Due to magnetic attraction, the silica-coated beads are attached to the magnet surface and appear as a brown dust. Three different buffer sets were tested.

Fecal sample prep buffer set 1:
a. Lysis buffer: 3 M guanidinium thiocyanate, 0.5 M NaCl, 0.1 M sodium acetate, pH 5.5, 2.5% Tween-20, 2.5% CHAPS, 10 mM EDTA;
b. Wash buffer: 70% acetone; and
c. Elution buffer: DNase/RNase-free water.

Fecal sample prep buffer set 2:
a. Lysis buffer: 3 M guanidinium thiocyanate, 0.5 M NaCl, 0.1 M sodium acetate, pH 5.5, 2.5% Tween-20, 2.5% n-octyl-B-D-glucopyranoside, 10 mM EDTA;
b. Wash buffer: 70% ethanol; and
c. Elution buffer: DNase/RNase-free water.

Fecal sample prep buffer set 3:
a. Lysis buffer: 3 M guanidinium thiocyanate, 0.5 M NaCl, 0.1 M sodium acetate, pH 5.5, 2.5% Tween-20, 2.5% n-octyl-B-D-glucopyranoside, 10 mM EDTA;
b. Wash buffer: 70% acetone; and
c. Elution buffer: DNase/RNase-free water.

The magnetic stick was then removed from the tube and inserted into a separate tube containing 950 μL of wash buffer. After 10 seconds, the magnetic stick was removed from the tube and allowed to air dry for approximately 2 minutes. The stick was then transferred to a separate tube containing 30 μL elution buffer and incubated for 5 minutes. Finally, the magnetic stick was added to a tube with elution buffer (30 μL of water) for 5 minutes at room temperature. Afterwards, the magnetic stick with bound silica beads was discarded. The elution buffer containing the eluted DNA was then added to target gene amplification reagents for the amplification of the invA gene in a PCR tube (See example 9). The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument or portable target gene amplification reader.

### Results

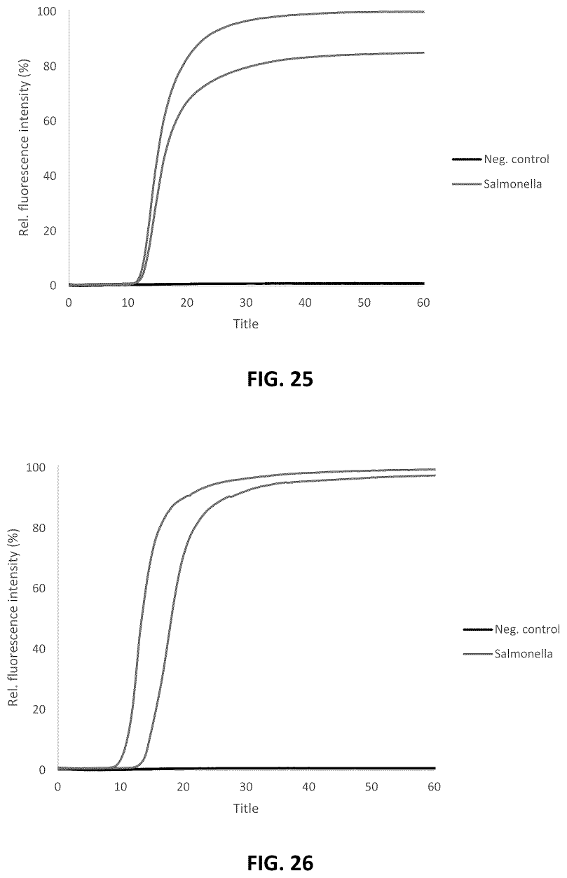

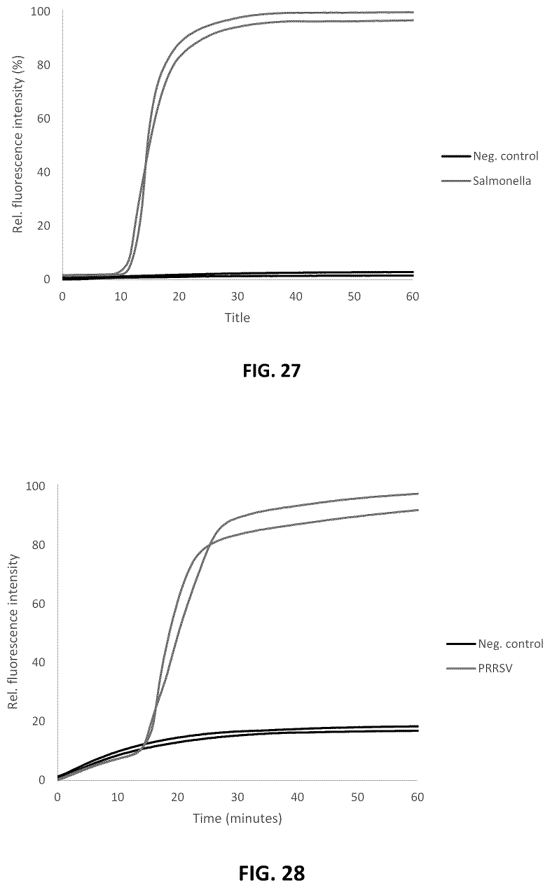

FIGS. 25 to 27 show fluorescence intensity graphs of the amplification of Salmonella in fecal samples prepared by the magnetic stick/bead method using three different buffer sets. A negative control was included that did not contain a nucleic acid target (black lines) and showed no amplification. The magnetic bead and stick method enabled rapid detection of Salmonella by DNA amplification after sample processing with buffer set 1 (FIG. 25), buffer set 2 (FIG. 26) and buffer set 3 (FIG. 27). These data confirm that POC amplification of a bacterial target from a very complex and biological matrix is possible using simple disposable supplies and a portable target gene amplification reader.

### Example 10: PRRSV in Swine Serum (Magnetic Bead+Stick Method)

Swine serum (200 uL) was added to a tube with lysis buffer (570 uL), and silica-coated magnetic beads (20 uL of a 50% slurry). Two different lysis buffers were tested.

PRRSV sample prep buffer set 1:
a. Lysis buffer: 100 mM sodium acetate, pH 5.5, 4 M guanidine thiocyanate, 10% ethanol;
b. Wash buffer: Ethanol; and
c. Elution buffer: DNase/RNase-free water.

PRRSV sample prep buffer set 2:
a. Lysis buffer: 100 mM Tris, pH 8.0, 4 M guanidine thiocyanate, 10% ethanol;
b. Wash buffer: Ethanol; and
c. Elution buffer: DNase/RNase-free water.

The magnetic stick was added to the lysis tube and incubated for 5 minutes at room temperature. After the incubation, the magnetic stick was removed from the lysis tube. Due to magnetic attraction, the silica-coated beads are attached to the magnet surface and appear as a brown dust. The stick was then added to a tube with wash buffer (950 uL of 70% ethanol) and incubated for 10 seconds. After washing, the stick was allowed to air dry for approximately 2 minutes. Finally, the stick was added to a tube with elution buffer (30 uL of water) for 5 minutes at room temperature. Afterwards, the stick with bound silica beads was discarded.

The elution buffer containing the eluted RNA was then added to target gene amplification reagents for the amplification of PRRS virus ORF 7 (see Example 1) in a PCR tube. The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument.

### Results

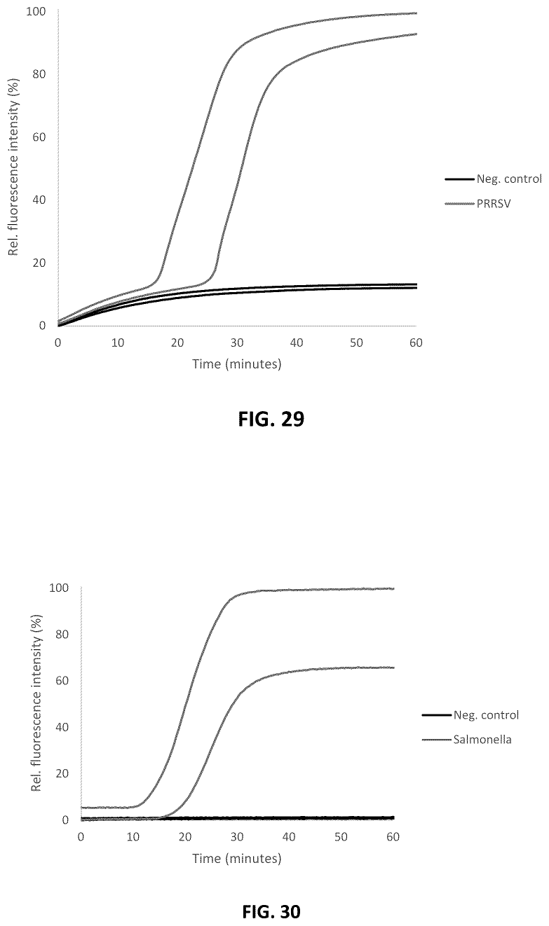

FIGS. 28 and 29 show fluorescent intensity curve graphs for the amplification of viral RNA from swine PRRS virus using the magnetic beads and stick method. Target gene amplification reactions were assembled from viral RNA prepared by the magnetic stick method (blue lines). Both lysis buffer set 1 (FIG. 28) and lysis buffer set 2 (FIG. 29) showed rapid amplification of PRRS virus genomic RNA, however set 1 showed better reproducibility in replicate tests. A negative control was included that contained water instead of a nucleic acid target (black lines). The magnetic bead and stick method may be particularly useful when testing sera with high levels of hemolysis or elevated lipids. These contaminants may interfere with target gene amplification reactions, so removal using the magnetic stick method would ensure no loss of sensitivity when testing for this deadly pathogen. These data also indicate that the magnetic stick method may enable the detection of PRRS virus from complex processing fluids (fluids from pooled testicle and tail tissues removed from young pigs) that require extensive nucleic acid purification for detection of PRRS virus using PCR methods.

### Example 11: Salmonella from Environmental Surfaces (Magnetic Bead+Stick Method)

Environmental samples taken from food processing plants may be obtained using sponges, cloths, dusters, or swabs that may require varying volumes of liquid to ensure elution of the trapped bacteria. Because of this need for large volume and the low number of organisms per mL present when sampling cleaned surfaces, most testing for Salmonella utilizes culture methods that take a minimum of 8 hours before testing for nucleic acids by amplification. The magnetic stick method has the advantage that the beads can capture nucleic acids from a large volume and elute into a small volume for amplification. Therefore, this method could improve the sensitivity of environmental sampling to eliminate or reduce the time needed for culture amplification. To determine if the magnetic stick method was compatible with environmental sampling, Salmonella was spiked onto surfaces, sampled with swabs, and processed for target gene amplification.

### Materials and Methods

A sample of Salmonella enterica subsp. enterica serovar Typhimurium (10 μL of sample for 100 cfu total) in Tris-buffered saline was applied to a stainless steel metal surface and allowed to dry at room temperature. A polyurethane swab wetted with a neutralization buffer (World Bioproducts PurBlue swab with HiCap™ Neutralizing Broth) was used to collect Salmonella from the metal surface. The swab was inserted into a lysis tube containing 3 mL lysis buffer (100 mM sodium acetate, pH 5.5, 3 M guanidine thiocyanate, 0.5 M sodium chloride, 2.5% Tween-20, 2.5% n-octyl-B-D-glucopyranoside) and 50 uL magnetic silica beads (BioChain PureSil). After incubating the swab in the lysis buffer for 5 minutes at room temperature, the swab was removed and discarded. A magnetic stick was added to the lysis tube and swirled around for approximately 10 seconds until all the beads are visibly bound to the magnetic stick. Due to magnetic attraction, the silica-coated beads are attached to the magnet surface and appear as a brown dust. The magnetic stick was then removed from the tube and lightly mixed in a separate tube containing 1 mL of wash buffer (70% ethanol). The stick was then removed from the wash buffer and allowed to air dry (approximately 2 minutes at room temperature). Once dry, the magnetic stick was transferred to a separate tube containing 100 μL elution buffer (0.1 mM EDTA) and incubated for 5 minutes. Finally, the magnetic stick was added to a tube with elution buffer (30 μL of water) for 5 minutes at room temperature. Afterwards, the magnetic stick with bound silica beads was discarded. The elution buffer containing the eluted DNA was then added to target gene amplification reagents in a PCR tube with invA primers (see Example 9). The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument or portable target gene amplification device.

### Results

FIG. 30 shows a fluorescent intensity graph of the amplification of genomic DNA from Salmonella obtained from the metal surface and prepared by the magnetic stick/bead method. target gene amplification reactions were assembled from genomic DNA prepared by the magnetic stick method. A negative control was included that did not contain a nucleic acid target (black lines). The magnetic bead and stick method enabled rapid detection of bacterial genomic DNA. This Example demonstrates that environmental samples collected by a swab and prepared by the magnetic bead and stick method enables rapid sample processing and sensitive detection in a target gene amplification assay.

### Example 12: Workflow for Magnetic Stick and Magnetic Comb

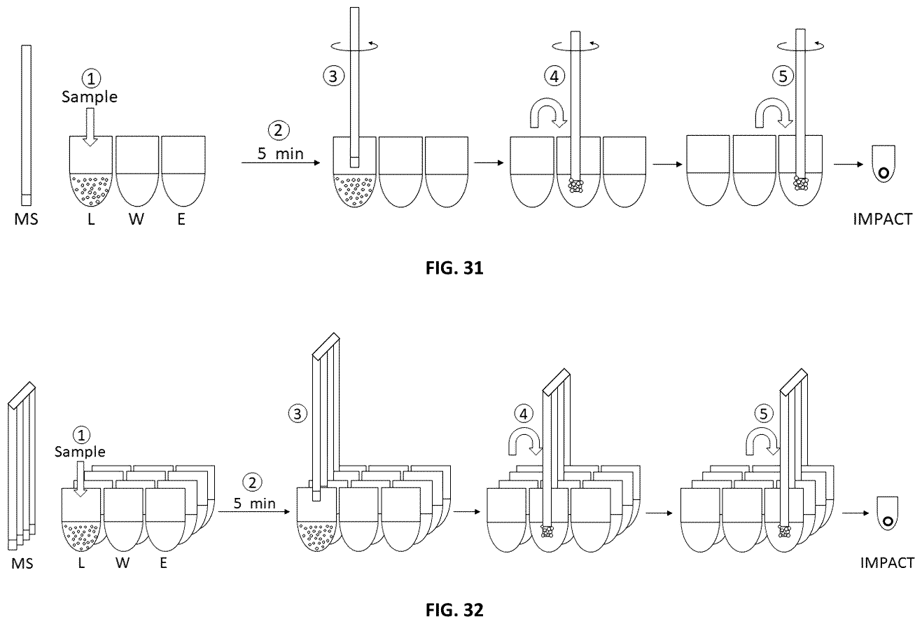

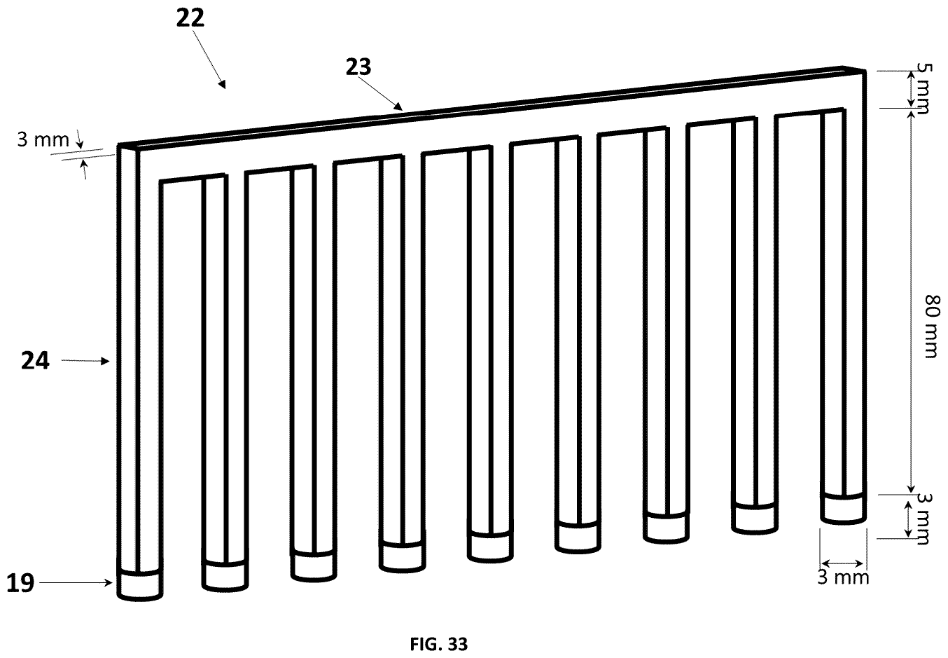

A commercial workflow is diagramed in FIGS. 31 and 33 for the preparation of nucleic acids from various samples for application to food safety and other sample testing using a magnetic stick (FIG. 31) and for the preparation of multiple samples in parallel using a magnetic comb (FIG. 32). Step 1 involves addition of a collected sample to a tube containing lysis buffer and silica-coated magnetic beads. The sample is incubated in lysis buffer/magnetic beads for about 5 minutes at room temperature (Step 2). A magnetic stick (MS) or magnetic comb is inserted into the lysis tube and beads are collected by mixing (Step 3). The stick or magnetic comb is transferred to the wash tube (W), where the magnetic stick or magnetic comb is mixed for about 10 seconds (Step 4). After air-drying the stick for up to approximately 2 minutes, the magnetic stick or magnetic comb is inserted into the elution tube (E) for a 10-minute incubation (Step 5). After the incubation, the magnetic stick or magnetic comb is discarded and the elution may be used in a downstream amplification reaction.

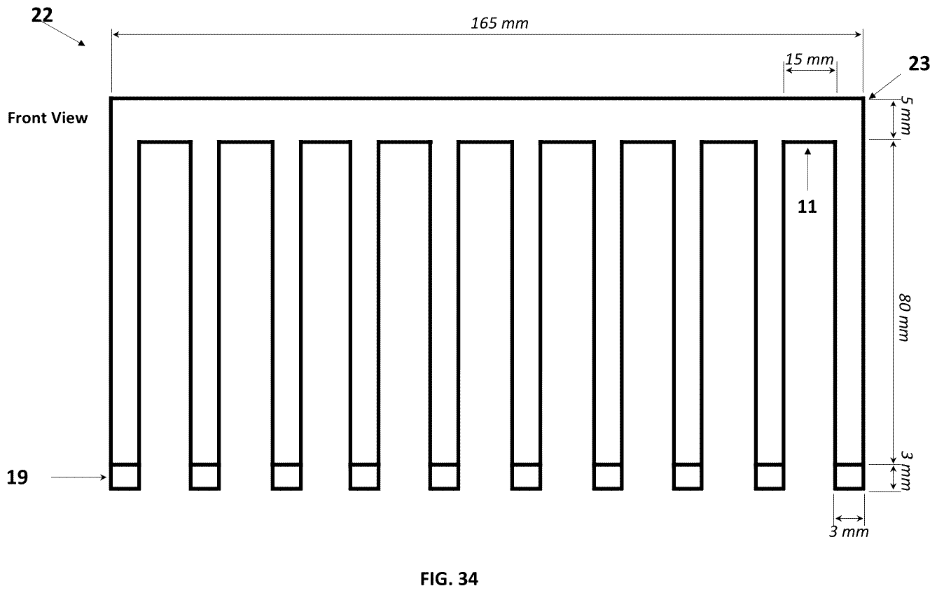

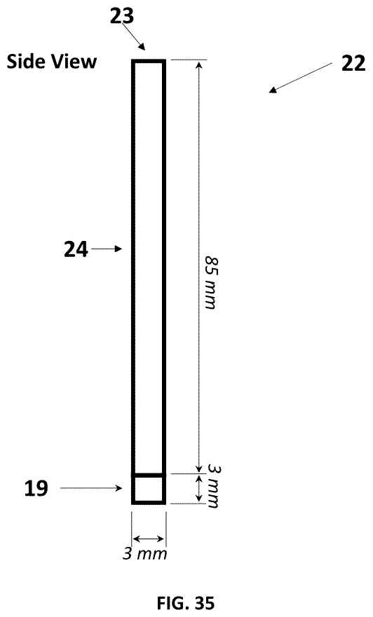

Details of the magnetic comb design that facilitates simultaneous capture of beads from lysis buffer, washing, and elution from multiple samples simultaneously are detailed in FIGS. 33 to 35. Following the sample preparation procedure, the elution containing the nucleic acids is transferred to a tube containing target gene amplification enzymatic reagents and primers and inserted into a device capable of heating (around 65° C.) and measuring fluorescence.

### Example 13: Detection of E. coli in Whole Milk (Direct Sample Addition Method)

### Materials and Methods

A sample of Escherichia coli (E. coli) (10 μL; 10⁵ cfu) was diluted in whole milk (90 μL). The diluted sample was lysed by heat (95° C. for 10 minutes) then added directly to liquid target gene amplification reagents in a PCR tube, including primer sequences from Table 6. Polyaspartic acid (PLD10; MW 1,400) was also added (0.05% w/v) to some of the target gene amplification reactions. The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument.

TABLE 6
Sequences of uidA primer set used to detect E.
coli in a target gene amplification reaction.

| | SEQ |
|---|---|
| Sequences | ID NO. |
| CAGGCGATTAAAGAGCTGAT | 52 |
| TGATGGTATCGGTGTGAGC | 53 |
| GGACGGGTATCCGGTTCGTTTGACAAAAACCACCCAAGC | 54 |
| ACGGGAATATTTCGCGCCACTTACATTGACGCAGGTGATC | 55 |
| GGCGGAAGCAACGCGTAAA | 56 |

An alternative primer set for the detection of the E. coli uidA genetic target is provided in Table 7. The uidA consensus sequence was compared to 1,000 separate E. coli strains using BLAST. Changes from the original sequences that target gene amplification primer sequences are shown in bold, underlined, and enlarged letters.

TABLE 7
Alternative sequences of a primer set for the
detection of E. coli uidA gene.

| | SEQ ID |
|---|---|
| Sequences | NO |
| CAGGCGATTAACGAGTTGAT | 57 |
| TGATGGTATCGGCGTGAGC | 58 |
| GGACGGGTATCCGGTTCGTTTGACAAAAACCACCCAAGC | 54 |
| ACGGGAATATTTCGCGCCACTTACATTGACGCAGGTGATC | 55 |
| GGCGGAAGCAACGCGTAAA | 56 |

### Results

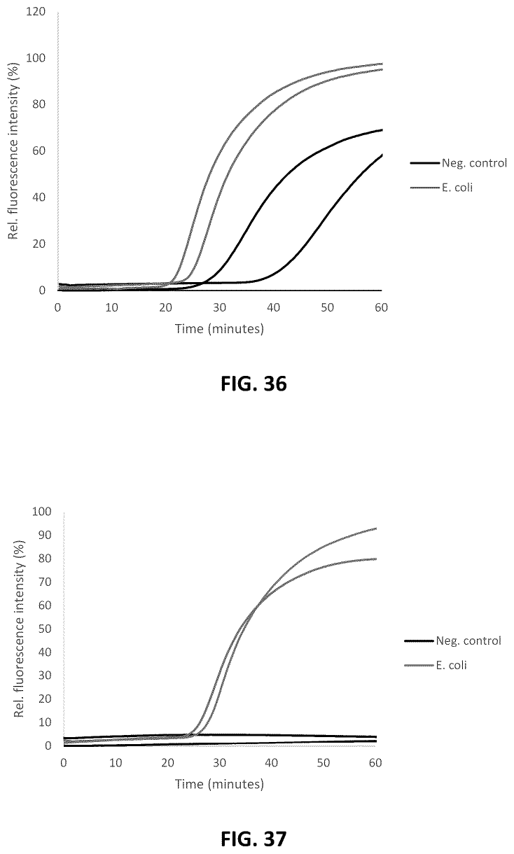

FIG. 36 is a fluorescent intensity curve graph showing the detection of E. coli in whole milk samples where target gene amplification reactions did not contain the additive PLD10 using the direct sample addition method. E. coli was spiked into a sample of whole milk and lysed by heat (blue lines). A negative control was included that contained milk without E. coli (black lines). The rapid amplification in the negative control would interfere with the detection of lower concentrations of E. coli in a milk sample.

FIG. 37 shows that target gene amplification reactions directly from milk containing the additive PLD10. E. coli was spiked into a sample of whole milk and lysed by heat (blue lines). A negative control was included that contained milk without E. coli (black lines). The addition of PLD10 prevented the false positive reactions in the negative control.

### Example 14: Capture and Concentration of Bacteria Using Anion Exchange Resin

An alternative way to capture bacteria using magnetic beads was explored for use with samples that may contain low amounts of bacteria per mL of sample.

### Materials and Methods

To a tube was added 20 μL of anion exchange resin slurry (75% slurry of a quaternary amine resin). The slurry was centrifuged and the supernatant was discarded to remove excess storage buffer components. A 100 μL sample of DH5alpha E. coli (10⁶ cfu total) was added to the beads and incubated at room temperature for 5 minutes. The tube was then centrifuged at a low speed that would not pellet E. coli and the supernatant discarded without disturbing the resin. A basic elution buffer (30 μL of 18.67 mM NaOH) was added to lyse the bacteria attached to the anion exchange resin. The sample was then centrifuged and the supernatant containing lysed bacteria and genomic DNA was added directly to a PCR tube containing target gene amplification reagents and uidA primers (see Example 14). The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument or portable target gene amplification device.

### Results

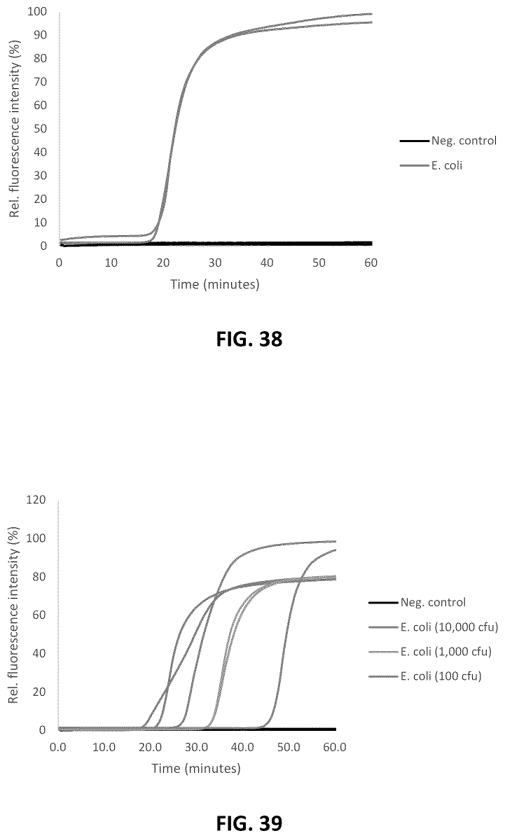

Anion exchange resin was used to capture bacteria from a mixture of growth media and E. coli (FIG. 38, red lines). A negative control was included which used anion exchange resin incubated in growth media without bacteria (black lines). Amplification only occurred when anion exchange resin was incubated with E. coli (10⁶ cfu total incubated with the anion exchange resin per experiment). This preliminary experiment indicates that anion exchange resin coated onto magnetic beads could be used to concentrate bacteria from large volume samples using magnetic sticks and thus eliminating the need for centrifugation.

### Example 15: Capture and Concentration of Bacteria Using a Syringe Filter

Methods to concentrate bacteria from dilute, large volume samples can circumvent the need for culture amplification, which takes at least 8 hours. Capture of bacteria on filters designed to sterilize liquids was explored as a way to concentrate bacteria from large volumes for detection by DNA amplification.

### Materials and Methods

A total of 0.1 mL of three different E. coli cultures were added to 9.9 mL of water to achieve final amounts of 10,000 cfu, 1,000 cfu, and 100 cfu. The solutions were then loaded into a 10 mL syringe and connected to a 0.22-micron syringe filter (25 mm diameter). Plungers were manually depressed and visually confirmed to fully pass all fluid through the syringe filter. Next, the syringe filter was transferred to a syringe loaded with 0.1 mL of lysis buffer (10 mM HCl, pH 2.1). The plunger on the second syringe was depressed to push the lysis buffer was passed through the syringe filter. The lysis buffer was expected to release DNA from trapped bacterial cells that are trapped on the filter. The free gDNA should pass through the filters because of its small size. The lysis buffer containing eluted genomic DNA was directly added to a PCR tube with liquid target gene amplification reagents including E. coli specific primers. The PCR tube was then inserted into an instrument that can maintain 65° C. and measure fluorescence every 20 seconds for 2 hours (excitation 470-490 nm, emission 510-530 nm). For example, a Bio-Rad CFX96 qPCR instrument or portable target gene amplification device.

### Results

FIG. 39 shows amplification of E. coli DNA from filtered samples that contained 10,000 cfu (red lines), 1,000 cfu (green lines), and 100 cfu (blue lines). A negative control was also included, which was filtered water without any E. coli added (black lines). These data indicate that the syringe filter captured E. coli and the lysis buffer successfully released genomic DNA from the captured cells that could be amplified by target gene amplification. Thus, this method can be refined for improving the sensitivity of target gene amplification POC tests by concentrating bacteria from dilute solutions into a small volume without the need for laboratory instruments like centrifuges.

### Example 16: Detection of Bacteria from Produce Wash (Direct Addition Method)

### Materials and Methods

To a piece of romaine lettuce was added 10 μL of a saturated overnight culture of E. coli and allowed to dry at room temperature (approx. 10 minutes). The E. coli was collected from the surface of the lettuce leaf using a cotton swab. The swab was swirled into 100 μL of water to release E. coli from the cotton swab. The water elution was then heated to 95° C. for 10 minutes to lyse the E. coli before adding the sample directly to a pre-assembled liquid target gene amplification reaction tube containing E. coli primers. The reaction was incubated in an instrument capable of heating to 65° C. while measuring fluorescence. For example, a Bio-Rad CFX96 qPCR instrument or portable target gene amplification device.

### Results

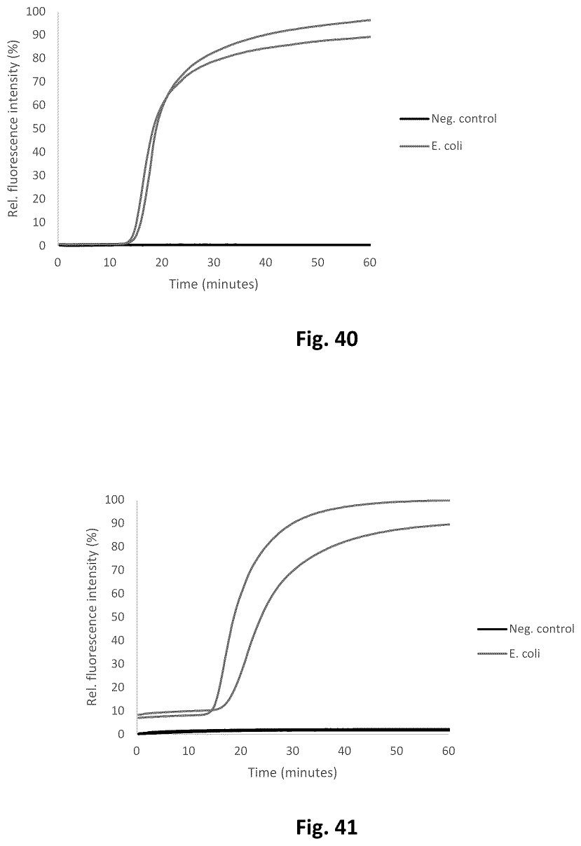

E. coli spiked onto the surface of a lettuce leaf was detected from a swab that was processed in ˜10 minutes (FIG. 40). Genomic DNA released from the crude heat lysis was readily detected in <20 minutes (blue lines). A negative control was also included, which contained only water spiked onto the surface of the lettuce leaf (no nucleic acid, black lines). These data confirm that a POC test for E. coli contamination of fruits and vegetables in less than 30 minutes is possible. Water collected from washed fruits or vegetables could be concentrated using the methods exemplified above and combined with this method to rapid detect E. coli with high sensitivity.

### Example 17: Detection of Bacteria in Peanut Butter (Direct Addition Method)

### Materials and Methods

Peanut butter was smeared onto a stainless-steel metal surface. To this smear was added 10 μL of a saturated overnight culture of E. coli. The peanut butter and E. coli mixture was collected from the metal surface using a cotton swab. The swab was swirled into 100 μL of water to release E. coli from the cotton swab. The water elution was then heated to 95° C. for 10 minutes to lyse the E. coli before adding the sample directly to a pre-assembled target gene amplification reaction incubated in an instrument capable of heating to 65° C. while measuring fluorescence. For example, a Bio-Rad CFX96 qPCR instrument or portable target gene amplification device.

### Results

E. coli spiked onto peanut butter smeared on a stainless-steel metal surface was detected from a swab that was processed in ˜10 minutes (FIG. 41). Genomic DNA released from the crude heat lysis was readily detected within about 20 minutes (blue lines). A negative control was also included, which contained only water spiked onto the peanut butter smear (no nucleic acid, black lines). The peanut butter components did not affect the target gene amplification reaction, despite being visually present in the target gene amplification reaction tube. These data confirm that a POC test for rapid detection of bacteria in complex food products is possible using target gene amplification.

### Example 18: Detection of Genomic RNA from Highly Pathogenic Avian Influenza

Influenza viruses can infect both birds and mammals of many types. Highly pathogenic avian influenza has caused repeated pandemics in chicken, turkey, and other birds raised for food around the world. The virus is believed to be spread from migrating birds to confined birds, but POC tests to rapidly detect the virus in wild or domestic birds are not available to confirm the sources of contamination or trace the virus spread.

### Materials and Methods

Genomic RNA from H5N1 avian influenza was purified and diluted 10-fold with water. The diluted RNA was added to a pre-assembled target gene amplification reaction containing H5N1 primer set (Table 8) and incubated in an instrument capable of heating to 65° C. while measuring fluorescence. For example, a Bio-Rad CFX96 qPCR instrument or portable target gene amplification device.

TABLE 8
Sequences of the H5N1 influenza A primer set.

| | SEQ ID |
|---|---|
| Sequences | NO. |
| TTAGCGGGCAATTCATCTC | 59 |
| GTGAGGGCTTCTGTCTTTG | 60 |
| ACAAACACATCCCCCCTGGAATTTGCCCCATTAGCGGAT | 61 |
| CTCATGCTCCCACTTGGAATGCCCATTGGAGTGCTTGTCAT | 62 |
| CCGATCCTTATACTGTTGTCCTTA | 63 |
| ACTCAGGGAGCCTTGCTGA | 64 |

### Results:

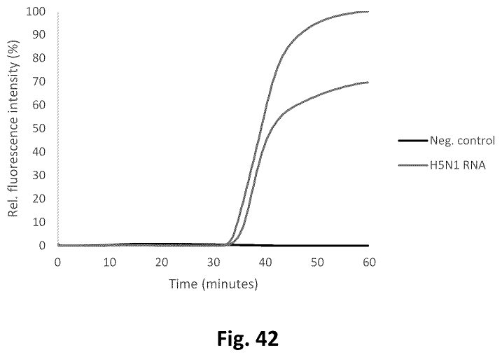

FIG. 42 show fluorescent intensity graphs of the amplification of viral RNA obtained from H5N1 influenza virus. As expected, the primer set did not generate fluorescence in the absence of nucleic acid sequences (negative control, black lines). The H5N1 primer set enabled amplification of the purified genomic RNA from H5N1 influenza virus (blue lines). These data when combined with other embodiments herein for rapid sample processing of virus samples, suggest that a POC rapid test for this virus is feasible.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A point-of-care/point-of-contact (POC) diagnostic system for detecting a gene-of-interest comprising:
a) a sample obtained from a material-of-interest;
b) a strand displacement DNA polymerase enzyme;
c) reagents, buffers, diluents, serums, enzyme co-factors, and dNTPs;
d) a set of at least 5 primers specific to the gene-of-interest;
e) a heating and reader device and;
f) data management software;
wherein the POC diagnostic system completes target gene amplification and detection within 90 minutes of sample collection; and
wherein the sample is optionally processed in less than four steps using additives, but without the purification of nucleic acids.

2. The POC diagnostic system of claim 1, further comprising a magnetic stick or magnetic comb for processing the sample by binding nucleic acids released from the sample, wherein the magnetic stick or magnetic comb comprise a body and a magnet, and wherein the body is bound to the magnet on the distal end of the body.

3. The POC diagnostic system of claim 1, further comprising a reverse transcriptase enzyme that catalyzes RNA-DNA conversion at temperatures between 60-70° C.

4. The POC diagnostic system of claim 1, wherein the material-of-interest is obtained from humans, non-human animals, plants, food, and water, and wherein the sample obtained from the material-of-interest is selected from a group consisting of serum, plasma, feces, urine, blood, oral fluids, bile, milk, colostrum, nasal secretions, oral secretions, ocular secretions, and fluids from tissues derived human, animal, other multicellular, complex species at risk for microbial diseases, minced, ground, mashed or similarly processed meat, fruits, vegetables, fish, bottled beverages, other processed food products with a risk of contamination with microbes, environmental surface at risk from contamination by infected animals or foods, a sample of water used to washed animals, foods, or environmental surfaces, or an enriched culture derived from the aforementioned samples.

5. The POC diagnostic system of claim 1, wherein the gene-of-interest is a marker of genetic modification that can determine species of origin for specific genetically-modified plants or animals that are used to produce human food.

6. The POC diagnostic system of claim 1, wherein the gene-of-interest is a marker of a microbial pathogen selected from a group consisting of a virus, bacterium, archaea, fungus, parasite or a microbial pathogen carrying a gene(s) facilitating resistance to a chemotherapeutic agent or antimicrobial drug used for treatment of disease, wherein the microbial pathogen infects a host's tissues and causes disease a host mammal, animal, insect, or plant or is a risk to cause disease in humans from contamination of food harvested from the host.

7. The POC diagnostic system of claim 6, wherein the virus is selected from porcine reproductive and respiratory syndrome virus (PRRSv), swine influenza virus, or avian influenza virus and wherein the bacterium selected from Salmonella enterica and it's subspecies and serovars, Listeria, and E. coli.

8. The POC diagnostic system of claim 1, wherein the reagents and enzymes are dried in one location of a test tube and the primers are dried in a separate location of said test tube, and wherein the enzymes and primers are dried under vacuum at room temp for 4 h to 24 h or under in a dry heat oven set at 40° C. to 60° C. and incubated for up to 2 hours or by freeze-drying.

9. The POC diagnostic system of claim 1, wherein the reagents are modified to reduce salts after sample addition in order to achieve a concentration under 100-120 mM and wherein the buffers and diluents for use with samples are modified to ensure pH ˜8.8 with detergents such as Tween or Triton added to the high pH lysis buffer.

10. The POC diagnostic system of claim 1, wherein the serum is heated to 70° C. to 80° C. for 3 to 10 minutes to release pathogen nucleic acids and inhibit nucleases or other biomolecules that may be present in complex samples, and wherein the serum sample is treated with a high pH lysis buffer comprising sodium hydroxide or potassium hydroxide at pH >11 to facilitate release of nucleic acids from a microbe and inhibit nucleases or other biomolecules that may be present in complex samples.

11. The POC diagnostic system of claim 1, wherein the primers are optimized to avoid primer-dimer pairs, hairpin loops, and other secondary nucleic acid interactions that limit DNA binding and/or polymerase recognition, and wherein the primer sets are selected from a group consisting of:
a) a set of primers having 99% sequence identity of SEQ ID NO: 1, 2, 3, 4, 5, 6, 7, 8, 9, 10, 11, 12, or the combination thereof targets a ORF7 genetic target of the PRRV genome,
b) a set of primers having 99% sequences identity to SEQ ID NO: 13, 14, 15, 16, 17, 18, 19, 20, 21, 22, 23, 24, 25, 26, 27, 28, 29, 30, 31, 32, 33, 34, 35, 36, 38, 39, 40, 41, 42, 43, 44, 45, 46, 47, 48, 49, 50, 51, or the combination thereof targets a invA of the Salmonella genome and can amplify Salmonella enterica strains Berta, Typhimurium, 4,[5],12:i, agona, and Braenderup;
c) a set of primers having 99% sequences identity to SEQ ID NO: 52, 53, 54, 55, 56, 57, 58 or the combination thereof targets a uidA gene of an E. coli genome; and
d) a set of primers having 99% sequences identity to SEQ ID NO: 59, 60, 61, 62, 63, 64, or the combination thereof targets H5N1 influenza A.

12. The POC diagnostic system of claim 2, wherein the body of the magnetic stick or the magnetic comb is comprised of a low density polyethylene, high density polyethylene, polypropylene, polyethylene terephthalate, polystyrene, polyvinyl chloride, polyoxymethylene, acrylic (polymethyl methacrylate), polyurethane, polycarbonate, polytetrafluoroethylene, polyetherimide, polyether ether ketone, nylon (polyamide), bamboo, wood, or metal (iron, aluminum, zinc, copper, magnesium, and alloys thereof).

13. The POC diagnostic system of claim 2, wherein the magnet of the magnetic stick or magnetic comb is comprised of neodymium, iron-nitride, tetraenite, hematite, magnetite, maghemite, alnico, ferrite, samarium cobalt, magnetic rubber, ilmenite, ulvospinel or electromagnet, and wherein the magnet attracts magnetic beads within the biological sample.

14. The POC diagnostic system of claim 1, wherein the heating block and reader device is a small and compact box that heats, incubates and reads at least 10 target gene amplification reactions at once.

15. A method for detecting a gene of interest in a material-of-interest comprising:
a) processing a sample for direct use in target gene amplification reactions;
b) generating a primer set targeted towards a conserved gene target;
c) drying reagents onto a test tube, wherein reaction reagents, DNA strand-displacing polymerase, and reverse transcriptase are dried at one location of a test tube and primers are dried at a second location of said test tube by heat or vacuum;
d) collecting a sample from the material-of-interest;
e) diluting the sample in buffers to achieve appropriate salt concentration and pH;
f) heating the sample to release pathogen nucleic acids and inhibit nucleases that may be present in the sample;
g) resuspending the dried reagents in the test tube using the treated sample;
h) inserting the test tube containing the treated sample into a heating and reader device capable of heating the sample at 65° C. and measuring fluorescence;
i) amplifying the DNA within 90 minutes; and
j) analyzing the measured fluorescence to detect the presence of the amplified DNA in the sample;
wherein the method is performed using 1 sample, 1 tube and is completed within 90 minutes.

16. The method of claim 15, wherein processing a sample for direct use in target gene amplification reactions comprising:
a) lysing the sample with a lysis buffer to release of nucleic acids from microbe;
b) binding the nucleic acids to silica-coated beads;
c) incubating the nucleic acid bound silica-coated magnetic beads with a magnetic stick to allow the binding of the silica-coated magnetic beads to the magnetic stick;
d) washing the beads on the magnetic stick to remove contaminants and residual components of the lysis buffer;
e) eluting of the nucleic acids bound to the magnetic stick into a buffer compatible with target gene amplification;
f) transferring the eluted nucleic acids to a tube containing dried target gene amplification reagents for DNA/RNA amplification;
wherein lysis of the sample and binding of the released nucleic acid to silica-coated beads are completed in a single step; and
wherein the sample is incubated with a lysis buffer combined with silica-coated beads and the sample matrix for 5 minutes at room temperature.

17. The method of claim 15, further comprising uncoating a virus with a high pH buffer.

18. The method of claim 15, further comprising a rapid lysis method for the direct addition of milk to target gene amplification reactions comprising;
a) lysing a milk-containing sample by heat;
b) adding the lysed sample to target gene amplification reagents in a PCR tube;
c) adding polyaspartic acid to the sample in the PCR tube; and
d) running a target gene amplification reaction;
wherein the milk is whole milk, reduced fat milk, skim milk, buttermilk, powdered milk, condensed milk, or evaporated milk.

19. The method of claim 15, further comprising a sample enrichment method comprising:
a) binding a sample to a positively charged anion resin;
b) separating the ion exchange resin by centrifugation, filtration, or magnetic force if bound to magnetic beads;
c) incubating the resin and bound sample in a lysis buffer to concentrate and extract DNA into a smaller volume;
d) adding the extracted DNA or RNA to target gene amplification reagents in a PCR tube; and
e) running a target gene amplification reaction.

20. The method of claim 15, further comprising a method of concentrating bacterial from dilute samples comprising:
a) loading a sample into a first syringe pre-connected to a first syringe filter;
b) depressing a plunger on the first syringe to force the liquid through the syringe, thereby trapping the sample on the syringe filter;
c) transferring the first syringe filter with the trapped sample to a second syringe containing a smaller volume of lysis buffer;
d) depressing the plunger on the second syringe to pass the lysis buffer through the first syringe filter, thereby passing the eluted sample genomic DNA through the filter; and
e) adding the eluted genomic DNA or RNA to a target gene amplification reaction for pathogen detection;
wherein the syringe filter has a 0.45, 0.22, or 0.1 filter comprising materials selected from a group consisting of nylon, polyethersulfone, cellulose acetate, regenerated cellulose, polypropylene, glass fiber, ceramic, metal, and wood; and
wherein the method is completed in 5 minutes or less.
